(19) United States
(12) Patent Application Publication
(10) Pub. No.: US 2026/0358332 A1
(43) Pub. Date: Nov. 12, 2026

(54) Particles

(71) Applicant: Life Technologies AS, Oslo (NO)

(72) Inventors: Daniel Sean Firth, Oslo (NO); Maren Teresa Johansen, Oslo (NO); Jan Christian Holgado Paulsen, Lillestrøm (NO); Geir Fonnum, Fjellhamar (NO); Muhammed Talha Gökmen, Oslo (NO)

(21) Appl. No.: 18/997,173

(22) PCT Filed: Jul. 21, 2023
(86) PCT No.: PCT/EP2023/070373

§ 371 Date: Jan. 20, 2025

(30) Foreign Application Priority Data
Jul. 22, 2022 (GB) 2210796.5

(51) Int. Cl. G01N 33/543; G01N 33/531

(52) U.S. Cl. CPC G01N 33/54326; G01N 33/531

(57) ABSTRACT

This invention relates to magnetic clusters comprising nanocrystals of iron oxide. The magnetic clusters comprise an aromatic carboxylic acid and/or the magnetic clusters are monodisperse. The magnetic clusters may be useful in biological assays and other applications. The invention also relates to processes for preparing such magnetic clusters and methods of using the magnetic clusters, as well kits comprising the magnetic clusters.

This invention relates to magnetic clusters comprising nanocrystals of iron oxide. The magnetic clusters comprise an aromatic carboxylic acid and/or the magnetic clusters are monodisperse. The magnetic clusters may be useful in biological assays and other applications. The invention also relates to processes for preparing such particles and methods of using the particles, as well as other subject matter.

### BACKGROUND

Particles, such as magnetic microparticles and magnetic nanoparticles are used in a wide variety of applications, such as biological assays and other applications. For example, nanoparticles and microparticles have been used in agglutination tests and assays, particle capture ELISA methods, lateral flow tests, solid-phase assays, scintillation proximity assays, polymerase chain reaction (PCR) tests, superparamagnetic-based assays and magnetic separation systems and biosensors; as enhancers of Raman spectral signals; in light-scattering assays; in drug delivery applications; and as fluorescent labels or stains for detecting biological molecules.

In some applications it is desirable to include magnetic material in the particles. This permits magnetic isolation of the polymeric particles, and use of the particles in superparamagnetic-based assays and magnetic separation systems and biosensors. It may also be desirable to use monodisperse particles, as monodisperse particles typically behave in a more consistent and reproducible manner in assays than polydisperse particles.

Magnetic microparticles and nanoparticles are often surface functionalized and/or coated prior to use in application. In addition, the known particles may be polymeric particles comprising magnetic material, such as porous polymeric particles comprising magnetic material in the pores. Examples of such particles include magnetic Ugelstad type particles, such as those described in WO 00/61647 or WO 2010/125170.

Known magnetic microparticles and magnetic nanoparticles that are used in applications, such as biological assays, are subject to a number of limitations. For example, for environmental reasons it may be desirable to minimize the use of (non-biodegradable) plastic in microparticles and nanoparticles. In addition, it may also be desirable to provide particles with a low CV. There is therefore a need for new microparticles and nanoparticles.

It is an aim of the invention to provide magnetic clusters comprising nanocrystals of iron oxide that are useful in applications, such as biological assays.

### BRIEF SUMMARY OF THE DISCLOSURE

The invention is based in part on an appreciation that the formation of magnetic nanoclusters, without the presence of significant polymer, can reduce the level of microplastics, providing a more environmentally compatible product. The invention is also based on an appreciation that monodisperse magnetic nanoclusters, when suitably functionalized, are useful in many different applications.

In accordance with a first aspect there is provided magnetic clusters, wherein the magnetic clusters comprise nanocrystals of iron oxide and an aromatic carboxylic acid.

In an embodiment the aromatic carboxylic acid is a compound of formula (I):

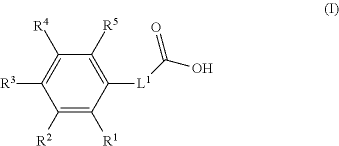

L¹ is selected from a bond, —C₁₋₆alkyl-, or —C₂₋₆alkenyl-. R¹ is selected from —H, —COOH, —(C₁₋₅alkyl)COOH, —(C₂₋₅alkenyl)COOH, or —C₁₋₄alkoxy. R² is selected from —H, -halo, —C₁₋₄alkyl, —C₁₋₄alkoxy, or —NO₂. R³ is selected from —H, -halo, —C₁₋₁₂alkyl, —C₃₋₈cycloalkly, —C₁₋₁₂alkoxy, -halo(C₁₋₁₂)alkyl, —NR⁶R⁷, —NO₂, —C(O)OH, —C(O)OR⁶, or substituted or unsubstituted phenyl. R⁴ is selected from —H, -halo, —C₁₋₄alkyl, —C₁₋₄alkoxy, or —NO₂. R⁵ is selected from —H, or —C₁₋₄alkoxy. R⁶ is selected from —H, or —C₁₋₄alkyl. R⁷ is selected from —H, —C₁₋₄alkyl, —C(O)H, —C(O)C₁₋₄alkyl. In another embodiment, R₁ and R₂ together with the carbon atoms to which they are attached form a substituted or unsubstituted phenyl, and L¹, R³, R⁴, R⁵, R⁶ and R⁷ are as defined previously. In another embodiment, R₂ and R₃ together with the carbon atoms to which they are attached form a substituted or unsubstituted phenyl, and L¹, R¹, R⁴, R⁵, R⁶ and R⁷ are as defined previously.

In an embodiment, L¹ is selected from a bond, —C₁₋₆alkyl-, or —C₂₋₆alkenyl-. R¹ is selected from —H, —COOH, —(C₁₋₅alkyl)COOH, —(C₂₋₅alkenyl)COOH, or —C₁₋₄alkoxy. R² is selected from —H, —C₁₋₄alkyl, —C₁₋₄alkoxy. R³ is selected from —H, -halo, —C₁₋₆alkyl, —C₁₋₆alkoxy, -halo(C₁₋₆)alkyl, —NR⁶R⁷, or substituted or unsubstituted phenyl. R⁴ is selected from —H, —C₁₋₄alkyl, —C₁₋₄alkoxy. R⁵ is selected from —H, or —C₁₋₄alkoxy. R⁶ is selected from —H, or —C₁₋₄alkyl. R⁷ is selected from —H, —C₁₋₄alkyl, —C(O)H, —C(O)C₁₋₄alkyl.

In an embodiment, L¹ is selected from a bond, —C₁₋₆alkyl-, or —C₂₋₆alkenyl-. R¹ is selected from —H, —COOH, —(C₁₋₅alkyl)COOH, —(C₂₋₅alkenyl)COOH, or —C₁₋₄alkoxy. R² is selected from —H, —C₁₋₄alkyl, —C₁₋₄alkoxy. R³ is selected from —H, —C₁₋₆alkyl, —C₁₋₆alkoxy, —NR⁶R⁷, or substituted or unsubstituted phenyl. R⁴ is selected from —H, —C₁₋₄alkyl, —C₁₋₄alkoxy. R⁵ is selected from —H, or —C₁₋₄alkoxy. R⁶ is selected from —H, or —C₁₋₄alkyl. R⁷ is selected from —H, —C₁₋₄alkyl, —C(O)H, —C(O)C₁₋₄alkyl.

A second aspect of the invention provides monodisperse magnetic clusters comprising nanocrystals of iron oxide, wherein the magnetic clusters have a mode diameter in the range of from about 100 nm to about 2,500 nm and a coefficient of variation (CV) of not more than 5%. The mode diameter and CV may be measured by disc centrifuge. The magnetic clusters further comprise an aromatic carboxylic acid, for example at least one aromatic carboxylic acid of formula (I).

A third aspect provides a method of making magnetic nanoclusters. The method comprises forming a solution comprising iron (III) ions, a protic solvent, an aromatic carboxylic acid, and a precipitator; and allowing the solution to react to form a suspension comprising magnetic clusters.

A fourth aspect provides magnetic clusters obtainable by a method of the third aspect.

A fifth aspect provides use of the magnetic clusters in an assay, wherein the magnetic clusters are of the first, second or third aspect.

A sixth aspect provides a kit comprising magnetic clusters of the first, second or third aspect. The kit may comprise one or more buffers.

### BRIEF DESCRIPTION OF THE DRAWINGS

Embodiments of the invention are further described hereinafter with reference to the accompanying drawings, in which:

FIG. 1 is a schematic illustrating a hypothesis for how magnetic clusters are formed from solution in the presence of citrate as a stabilizer.

FIG. 2 is an optical microscopy image of magnetic clusters, illustrating magnetic clusters at 5 different levels of agglomeration. The scale bar in the bottom right of each image represents 50 μm.

FIG. 3 provides optical microscopy images of magnetic clusters obtained with various stabilizers at aggregation levels 0 to 2. The scale bar in the bottom right of each image represents 50 μm.

FIG. 4 provides the particle size distribution for magnetic clusters formed using citric acid or toluic acid at a mol ratio of 0.2, 0.4, 0.8, or 1.6 as stabilizers.

FIG. 5 provides the particle size distribution for magnetic clusters formed using 3,4,5-trimethoxybenzoic acid (eudesmic acid), 4-acetamidonezoic acid, Biphenyl-4-carboxylic acid, Trans-cinnamic acid, 4-(dimethylamino)benzoic acid, 3,5-dimethylbenzoic acid, 4-ethylbenzoic acid, 4-t-butylbenzoic acid, benzoic acid, p-anisic acid, phthalic acid, homophthalic acid, or 3-phenylpropionic acid.

FIG. 6 illustrates the mode diameters of magnetic clusters (diamond markers) and CV % as measured by disc centrifuge.

FIG. 7 provides scanning electron microscopy (SEM) images comparing magnetic clusters prepared using bisphenyl-4-carboxylic acid as the stabilizer (left images) to those prepared using citrate as the stabilizer (right images).

FIG. 8 provides the particle size distribution for magnetic clusters formed using 4-(Trifluoromethyl)benzoic acid, 4-iodobenzoic acid, or 4-chlorobenzoic acid.

FIG. 9 illustrates, for the distributions of FIG. 7 the mode diameters of magnetic clusters (diamond markers) and CV % as measured by disc centrifuge.

FIG. 10 is an image of a gel illustrating the use of Protein G functionalised magnetic clusters for immunoprecipitation.

FIG. 11 provides a histogram showing the data from the measurement of cluster size by scanning electron microscopy (SEM) procedure provided herein. This data is based on the particles depicted in FIG. 7 and compares particles produced with bisphenyl-4-carboxylic acid (dark grey) and monosodium citrate (light grey).

FIG. 12 provides magnetic hysteresis curves obtained for magnetic clusters. (A) Provides the hysteresis curve for the magnetic clusters of Example 5 using a magnetic saturation of 72.64 Am²/kg. (B) Provides the hysteresis curve for magnetic clusters of Example 10 using a magnetic saturation of 72.27 Am²/kg.

### DETAILED DESCRIPTION

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

The term “aromatic carboxylic acid,” by itself or as part of another substituent, means, unless otherwise stated, a molecule or moiety comprising an aromatic group (e.g. phenyl) covalently bonded to a carboxylic acid group. The carboxylic acid group may be directly attached to the aromatic group, e.g. as per benzoic acid, or the carboxylic acid may be attached to the aromatic group via a linker.

“Magnetic” and “magnetic material” means responds to a magnetic field. When the magnetic material is paramagnetic, the magnetic properties are switched off when the magnetic field is removed. When the magnetic material is superparamagnetic, the magnetic material becomes saturated at relatively low magnetic fields and switching off of the magnetic properties with removal of the magnetic field is very rapid/instant. Paramagnetic and superparamagnetic materials may therefore be considered “magnetisable”, as the magnetic properties are dependent on the application of an external magnetic field. When the magnetic material is ferromagnetic, all of its magnetic atoms within each domain add a positive contribution to the net magnetization. When the magnetic material is ferrimagnetic, some magnetic atoms within each domain are opposed, but overall the material exhibits net magnetization. Both ferromagnetic and ferrimagnetic material retain magnetic properties after an external magnetic field is removed. Above the material's Currie temperature, ferromagnetic and ferromagnetic material becomes a paramagnetic material. The magnetic properties may also be affected by the size of the magnetic particles in the magnetic material, with some materials being ferromagnetic and ferrimagnetic at larger particle sizes, but superparamagnetic suitably small particle sizes (e.g. nm scale). For example, ferrimagnetic material, e.g. iron oxides, form superparamagnetic crystals when the size of the crystals is sufficiently small (e.g. below about 15 nm scale for iron oxides). Magnetic clusters of the disclosure and invention may be paramagnetic or superparamagnetic, as they comprise nanocrystals of iron oxide.

“Magnetic material precursor” means a substance that may be converted to provide a magnetic material. A magnetic material precursor may comprise solvated transition metal ions (e.g. polyvalent cations of Fe, Ni, Co or a combination thereof, optionally in admixture with polyvalent cations of Al, Mn, Cu, Zn, Ca, Ge, Te, Ti or Sn and/or rare earths). The solvated transition metal ions and/or rare earth ions may be converted to a magnetic material by any process that causes the ions to precipitate, e.g. as oxides. Precipitation may be caused by, for example, a pH change, removal of solvent, or a change in temperature. For example, a magnetic material precursor may be provided by an aqueous suspension of pH less than 6 comprising Fe²⁺ and/or Fe³⁺ ions; and the Fe²⁺ and/or Fe³⁺ ions may be converted to a magnetic material by precipitation, e.g. by raising the pH to more than 8.

“Nucleic acid” means a molecule made up of ribonucleotides and/or deoxyribonucleotides as well as synthetic nucleotide residues that are capable of participating in Watson-Crick type or analogous base pair interactions, i.e. “hybridisation” or the formation of a “duplex”. Thus, the nucleic acid may be single-stranded or double-stranded DNA or RNA or any modification thereof, including conformationally restricted or nucleobase analogue-bearing oligomers such as “locked-nucleic acids” (LNA) or “peptide nucleic acids” (PNA) or other derivatives containing non-nucleotide backbones. The nucleic acid may be a naturally-occurring molecule, i.e. DNA or RNA but also include DNA/RNA hybrids where the DNA is in separate strands or in the same strand) in which the 3′ position of the pentose of one nucleotide is joined by a phosphodiester linkage to the 5′ position of the pentose of the next nucleotide. Nucleic acids used in various embodiments may comprise chemically, enzymatically, or metabolically modified forms of nucleotides or combinations thereof, such as primers, probes, oligonucleotides or aptamers.

“Coating” means a covering that is applied to the surface of a substrate. The coating may be an all-over coating, completely covering the substrate, or it may be a partial coating, only covering a portion of the surface of the substrate. The coating may be applied to the substrate by any suitable type of bonding, for example by at least one of covalent bonding, metallic bonding, ionic bonding, hydrogen bonding, van der Waals interactions, hydrophobic interactions, and the like. Where the substrate comprises a magnetic material (such as magnetic clusters), the coating (or at least a part thereof) may be silica, which may formed on the surface of the magnetic material by condensation of silicates or orthosilicates. The coating may comprise an organic coating, which may comprise a spacer (such as an oligoethyleneglycol or polyethyleneglycol) and/or a polymer. A coating may comprise functional groups, such as hydroxyls, carboxylic acids, primary amines, secondary amines, or epoxys.

A “cluster” as used herein means a clump or particle composed of multiple smaller entities. For example, magnetic nanoclusters are nano-sized particles composed of or formed from multiple nanocrystals of iron oxide. Thus, in many instances the terms cluster(s) and particle(s) may be used interchangeably herein. Particles or clusters having a spherical shape are also often referred to as beads.

“Functional group” or “reactive group” means a substituent that is able to undergo characteristic chemical reactions. Exemplary functional groups/reactive groups of the present disclosure include hydroxyl groups, carboxylic acid groups, aldehyde groups, amine groups (e.g. primary or secondary aliphatic amine groups, and aromatic amine groups), thiol groups, epoxy groups, amide groups, chloromethyl groups, or tosyl-activated groups. Functional groups may be present on the surface of a substrate (such as magnetic clusters), for example functional groups may be present on a coating.

“Ligand” means a molecule that is able to bind to another species. A ligand may be a capture ligand, for example a capture ligand that is useful in an assay. Exemplary ligands include antibodies, antibody fragments, peptides, carbohydrates, haptens, aptamers, and oligonucleotides. Ligands may be bound to a substrate (such as magnetic clusters) comprising functional groups using standard techniques. Examples of such techniques are described in Chapter 14 of G. T. Hermanson, Bioconjugate Techniques, Academic Press, (3^{rd} Edition, 1996).

The term “antibody”, as used herein, includes: (a) any of the various classes or sub-classes of immunoglobulin (e.g., IgG, IgA, IgM, IgD or IgE derived from any animal e.g., any of the animals conventionally used, e.g., sheep, rabbits, goats, mice, camelids, or egg yolk), (b) monoclonal or polyclonal antibodies, (c) intact antibodies or fragments of antibodies, monoclonal or polyclonal, the fragments being those which contain the binding region of the antibody, e.g., fragments devoid of the Fc portion (e.g., Fab, Fab′, F(ab′)2, scFv, VHH antibodies, VHH antibody fragments, as well as other single domain antibodies), the so called “half molecule” fragments obtained by reductive cleavage of the disulphide bonds connecting the heavy chain components in the intact antibody (Fv may be defined as a fragment containing the variable region of the light chain and the variable region of the heavy chain expressed as two chains), and (d) antibodies produced or modified by recombinant DNA or other synthetic techniques, including monoclonal antibodies, fragments of antibodies, “humanized antibodies”, chimeric antibodies, or synthetically made or altered antibody-like structures.

The mention in this specification of “average” diameters unless otherwise specified refers to the mode diameter for magnetic clusters. The mode diameter may be measured by disc centrifuge, for example by a CPS disc centrifuge.

The term “monodisperse” means that for a plurality of particles (e.g. at least 100, more preferably at least 1000) the particles have a coefficient of variation (CV) or % polydispersity of their diameters of less than 20%, for example less than 15%, typically of less than 10% and optionally of less than 8%, e.g. less than 5%. The CV may be measured by disc centrifuge. CV is preferably calculated on the main mode, e.g. based on the width of the mode peak at half height. Thus some particles below or above mode size may be discounted in the calculation. Such a determination of CV is performable on a CPS disc centrifuge. An exemplary assay for measurement of particle size and CV for magnetic clusters of the disclosure by CPS disc centrifuge is provided herein below under the heading “Assays”.

The term “alkyl” as used herein include reference to a straight or branched chain alkyl moiety having, e.g. 1, 2, 3, 4, 5, 6, 7, 8, 9 or 10 carbon atoms. The term includes reference to, for example, methyl, ethyl, propyl (n-propyl or isopropyl), butyl (n-butyl, sec-butyl or tert-butyl), pentyl, hexyl and the like. In particular, alkyl may be a “C₁-C₆ alkyl”, i.e. an alkyl having 1, 2, 3, 4, 5 or 6 carbon atoms; or a “C₁-C₄ alkyl”, i.e. an alkyl having 1, 2, 3 or 4 carbon atoms. The term “lower alkyl” includes reference to alkyl groups having 1, 2, 3 or 4 carbon atoms. An alkyl may be optionally substituted, where chemically possible, by 1 to 5 substituents which are each independently at each occurrence selected from: oxo, ═NR^{a}, ═NOR^{a}, halo, nitro, cyano, NR^{a}R^{a}, NR^{a}S(O)₂R^{a}, NR^{a}CONR^{a}R^{a}, NR^{a}CO₂R^{a}, OR^{a}; SR^{a}, S(O)R^{a}, S(O)₂OR^{a}, S(O)₂R^{a}, S(O)₂NR^{a}R^{a}, CO₂R^{a} C(O)R^{a}, CONR^{a}R^{a}, C₁-C₄-alkyl, C₂-C₄-alkenyl, C₂-C₄-alkynyl, C₁-C₄ haloalkyl; wherein R^{a} is independently at each occurrence selected from: H and C₁-C₄ alkyl.

The term “alkoxy” as used herein include reference to —O-alkyl, wherein alkyl is straight or branched chain and comprises 1, 2, 3, 4, 5 or 6 carbon atoms. In one class of embodiments, alkoxy has 1, 2, 3 or 4 carbon atoms, e.g. 1, 2 or 3 carbon atoms. This term includes reference to, for example, methoxy, ethoxy, propoxy, isopropoxy, butoxy, tert-butoxy, pentoxy, hexoxy and the like. The term “lower alkoxy” includes reference to alkoxy groups having 1, 2, 3 or 4 carbon atoms.

The term “aromatic carboxylic acid” as used herein refers to an organic compound comprising a phenyl moiety substituted with at least one carboxylic acid moiety (e.g. —COOH, —(C₁₋₅alkyl)COOH, or —(C₂₋₅alkenyl)COOH). The aromatic carboxylic acid may also comprise further substituents, such as -halo, —C₁₋₆alkyl, —C₁₋₆alkoxy, -halo(C₁₋₆)alkyl, -halo(C₃₋₆)cycloalkyl, —NR⁶R⁷, —NO₂, —C(O)OH. The level of protonation of an aromatic carboxylic acid is dependent upon pH. Accordingly, unless the context requires otherwise, reference to an aromatic carboxylic acid includes reference to the acid, a salt form thereof, or a mixture of the acid and one or more salt forms thereof. Exemplary aromatic carboxylic acids of the disclosure are provided by compounds of formula (I).

The terms “halo” or “halogen,” as used herein include reference to a fluorine, chlorine, bromine, or iodine atom. The term “halide” as used as used herein include reference to a fluoride, chloride, bromide, or iodide ion. Additionally, terms such as “haloalkyl,” are meant to include monohaloalkyl and polyhaloalkyl. For example, the term “halo(C₁-C₆)alkyl” is meant to include, but not be limited to, trifluoromethyl, 2,2,2-trifluoroethyl, 4-chlorobutyl, 3-bromopropyl, and the like.

The term “phenyl” as used herein includes reference to a benzene ring. A phenyl may be optionally substituted, where possible, by 1 to 5 substituents which are each independently at each occurrence selected from: -halo, —C₁-C₆ alkyl, —C₂-C₆ alkenyl, —C₁-C₆ haloalkyl, and —C₂-C₆ haloalkenyl.

The term “substituted” as used herein in reference to a moiety means that one or more, especially up to 5, more especially 1, 2 or 3, of the hydrogen atoms in said moiety are replaced independently of each other by the corresponding number of the described substituents. Unless otherwise specified, exemplary substituents include —OH, —CN, —NH₂, ═O, -halo, —C₁-C₆ alkyl, —C₂-C₆ alkenyl, —C₁-C₆ haloalkyl, —C₁-C₆ haloalkoxy and —C₂-C₆ haloalkenyl, —C₁-C₆ alkylcarboxylic acid (e.g. —CH₃OOOH or —COOH). Where the substituent is a —C₁-C₆ alkyl or —C₁-C₆ haloalkyl, the C₁-C₆ chain is optionally interrupted by an ether linkage (—O—) or an ester linkage (—C(O)O—). Exemplary substituents for a substituted alkyl may include —OH, —CN, —NH₂, ═O, -halo, —CO₂H, —C₁-C₆ haloalkyl, —C₁-C₆ haloalkoxy and —C₂-C₆haloalkenyl, —C₁-C₆ alkylcarboxylic acid (e.g. —CH₃OOOH or —COOH). For example, exemplary substituents for an alkyl may include —OH, —CN, —NH₂, ═O, -halo.

It will, of course, be understood that substituents are only at positions where they are chemically possible, the person skilled in the art being able to decide (either experimentally or theoretically) without inappropriate effort whether a particular substitution is possible. For example, amino or hydroxy groups with free hydrogen may be unstable if bound to carbon atoms with unsaturated (e.g. olefinic) bonds. Additionally, it will of course be understood that the substituents described herein may themselves be substituted by any substituent, subject to the aforementioned restriction to appropriate substitutions as recognised by the skilled person.

Where steric issues determine placement of substituents on a group, the isomer having the lowest conformational energy may be preferred.

Where a compound, moiety, process or product is described as “optionally” having a feature, the disclosure includes such a compound, moiety, process or product having that feature and also such a compound, moiety, process or product not having that feature. Thus, when a moiety is described as “optionally substituted”, the disclosure comprises the unsubstituted moiety and the substituted moiety.

Where two or more moieties are described as being “independently” or “each independently” selected from a list of atoms or groups, this means that the moieties may be the same or different. The identity of each moiety is therefore independent of the identities of the one or more other moieties.

### Magnetic Clusters

The invention includes embodiments, wherein the magnetic clusters comprise nanocrystals of iron oxide and an aromatic carboxylic acid. The invention also includes embodiments wherein the magnetic clusters are monodisperse and comprise nanocrystals of iron oxide. Unless the context requires otherwise, reference to magnetic clusters below includes references to magnetic clusters of the invention that comprise an aromatic carboxylic acid and/or monodisperse magnetic clusters of the invention.

The aromatic carboxylic acid may be a compound of formula (I):

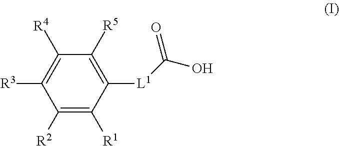

L¹ is selected from a bond, —C₁₋₆alkyl-, or —C₂₋₆alkenyl-. R¹ is selected from —H, —COOH, —(C₁₋₅alkyl)COOH, —(C₂₋₅alkenyl)COOH, or —C₁₋₄alkoxy. R² is selected from —H, -halo, —C₁₋₄alkyl, —C₁₋₄ alkoxy, or —NO₂. R³ is selected from —H, -halo, —C₁₋₁₂alkyl, —C₃₋₈cycloalkly, —C₁₋₁₂alkoxy, -halo(C₁₋₁₂)alkyl, —NR⁶R⁷, —NO₂, —C(O)OH, —C(O)OR⁶, or substituted or unsubstituted phenyl. R⁴ is selected from —H, -halo, —C₁₋₄alkyl, —C₁₋₄alkoxy, or —NO₂. R⁵ is selected from —H, or —C₁₋₄alkoxy. R⁶ is selected from —H, or —C₁₋₄alkyl. R⁷ is selected from —H, —C₁₋₄alkyl, —C(O)H, —C(O)C₁₋₄alkyl. Alternatively, R₁ and R₂ together with the carbon atoms to which they are attached form a substituted or unsubstituted phenyl, and L¹, R³, R⁴, R⁵, R⁶ and R⁷ are as defined herein. In another alternative, R₂ and R₃ together with the carbon atoms to which they are attached form a substituted or unsubstituted phenyl, and L¹, R¹, R⁴, R⁵, R⁶ and R⁷ are as defined elsewhere.

In an embodiment, L¹ is selected from a bond, —C₁₋₆alkyl-, or —C₂₋₆alkenyl-. R¹ is selected from —H, —COOH, —(C₁₋₅alkyl)COOH, —(C₂₋₅alkenyl)COOH, or —C₁₋₄alkoxy. R² is selected from —H, —C₁₋₄alkyl, —C₁₋₄alkoxy. R³ is selected from —H, halo, —C₁₋₆alkyl, —C₁₋₆alkoxy, -halo(C₁₋₆)alkyl, —NR⁶R⁷, or substituted or unsubstituted phenyl. R⁴ is selected from —H, —C₁₋₄alkyl, —C₁₋₄alkoxy. R⁵ is selected from —H, or —C₁₋₄alkoxy. R⁶ is selected from —H, or —C₁₋₄alkyl. R⁷ is selected from —H, —C₁₋₄alkyl, —C(O)H, —C(O)C₁₋₄alkyl.

L¹ may be selected from a bond, —C₁₋₄alkyl-, or —C₂₋₄alkenyl-. L¹ may be a bond. L¹ may be selected from —C₁₋₄alkyl-, or —C₂₋₄alkenyl-. L¹ may be —C₁₋₆alkyl-, optionally a —C₁₋₄alkyl-. L¹ may be —C₂₋₆alkenyl-, optionally a —C₂₋₄alkenyl-.

R¹ may be selected from —H, —COOH, —(C₁₋₅alkyl)COOH, or —(C₂₋₅alkenyl)COOH. R1 may be selected from —H, or —COOH. R¹ may be —H. R¹ may be —COOH. R¹ may be —H—(C₁₋₅alkyl)COOH. R¹ may be —(C₂₋₅alkenyl)COOH. R¹ may be —C₁₋₄alkoxy.

R² may be selected from —H, —C₁₋₄alkyl, or —C₁₋₄alkoxy. R² may be selected from —H, or —C₁₋₄alkoxy. R² may be selected from —H, or —OCH₃. R² may be —H. R² may be —C₁₋₄alkyl, e.g. R² may be —CH₃. R² may be —C₁₋₄alkoxy, e.g. R² may be —OCH₃.

R₁ and R₂ together with the carbon atoms to which they are attached may form a substituted or unsubstituted phenyl.

R³ may be selected from —H, -halo, —C₁₋₆alkyl, —C₃₋₆cycloalkly, —C₁₋₆alkoxy, -halo(C₁₋₆)alkyl, —NR⁶R⁷, —NO₂, —C(O)OH, or substituted or unsubstituted phenyl. R³ may be selected from —H, -halo, —C₁₋₆alkyl, —C₁₋₆alkoxy, -halo(C₁₋₆)alkyl, —NR⁶R⁷, or substituted or unsubstituted phenyl. R³ may be selected from —H, —C₁₋₄alkyl, -halo, —C₁₋₄alkoxy, -halo(C₁₋₄)alkyl, —NR⁶R⁷, or substituted or unsubstituted phenyl. R³ may be selected from —H, —C₁₋₄alkyl, —C₁₋₄alkoxy, -halo(C₁₋₄)alkyl, —NR⁶R⁷, or substituted or unsubstituted phenyl. R³ may be selected from —H, —C₁₋₄alkyl, —C₁₋₄alkoxy, —NR⁶R⁷, or substituted or unsubstituted phenyl. R³ may be selected from —C₁₋₄alkoxy, -halo(C₁₋₄)alkyl, —NR⁶R⁷, or substituted or unsubstituted phenyl. R³ may be selected from —C₁₋₄alkoxy, —NR⁶R⁷, or substituted or unsubstituted phenyl. R³ may be selected from —C₁₋₄alkoxy, -halo(C₁₋₄)alkyl, —NR⁶R⁷, or unsubstituted phenyl. R³ may be selected from —C₁₋₄alkoxy, —NR⁶R⁷, or unsubstituted phenyl. R³ may be selected from —H, —C₁₋₄alkyl, —Cl, —I, —OCH₃, —CF₃, —N(CH₃)₂, —NHC(O)CH₃, or phenyl. R³ may be selected from —H, —C₁₋₄alkyl, —OCH₃, —N(CH₃)₂, —NHC(O)CH₃, or phenyl. R³ may be selected from —OCH₃, —CF₃, —N(CH₃)₂, —NHC(O)CH₃, or phenyl. R³ may be selected from —OCH₃, —N(CH₃)₂, —NHC(O)CH₃, or phenyl.

R³ may be selected from —H, —C₁₋₁₀alkoxy, —NR⁶R⁷, —C(O)OR⁶, or substituted or unsubstituted phenyl. R³ may be selected from —H, —C₁₋₄alkyl, —OCH₃, —OCH₂(CH₂)₈CH₃, —N(CH₃)₂, —NHC(O)CH₃, —C(O)OCH₃, or phenyl.

R³ may be —H. R³ may be —C₁₋₆alkyl, e.g. R³ may be —C₁₋₄alkyl. R³ may be —C₁₋₆alkoxy, e.g. R³ may be —C₁₋₄alkoxy. R³ may be —OCH₃. R³ may be —NR⁶R⁷. R³ may be substituted or unsubstituted phenyl, e.g. R³ may be unsubstituted phenyl. R³ may be -halo, e.g. R³ may be —Cl. R³ may be -halo(C₁₋₆)alkyl, e.g. R³ may be -halo(C₁₋₄)alkyl. R³ may be —CF₃. R³ may be C₁₋₁₂alkyl, e.g. R³ may be —OCH₂(CH₂)₈CH₃ or —OCH₃. R³ may be —C(O)OR⁶, e.g. R³ may be —C(O)OCH₃.

R₂ and R₃ together with the carbon atoms to which they are attached may form a substituted or unsubstituted phenyl.

R⁴ may be selected from —H, —C₁₋₄alkyl, or —C₁₋₄alkoxy. R⁴ may be selected from —H, or —C₁₋₄alkoxy. R⁴ may be selected from —H, or —OCH₃. R⁴ may be —H. R⁴ may be —C₁₋₄alkyl, e.g. R⁴ may be —CH₃. R⁴ may be —C₁₋₄alkoxy, e.g. R⁴ may be —OCH₃.

In embodiments, not more than one of R², R³ and R⁴ may be a -halo.

R⁵ may be selected from —H, or —OCH₃. R⁵ may be —H. R⁵ may be —C₁₋₄alkoxy, e.g. R⁵ may be —OCH₃.

R⁶ may be —H or —CH₃. R⁶ may be —H. R⁶ may be —C₁₋₄alkyl, e.g. R⁶ may be —CH₃.

R⁷ may be —CH₃ or —C(O)CH₃. R⁷ may be —H. R⁷ may be —C₁₋₄alkyl, e.g. R⁷ may be —CH₃. R⁷ may be —C(O)H. R⁷ may be —C(O)C₁₋₄alkyl, e.g. R⁷ may be —C(O)CH₃.

The aromatic carboxylic acid may be selected from benzoic acid, toluic acid (e.g. p-toluic acid), 4-ethylbenzoic acid, 3,5-dimethylbenzoic acid, 4-t-butylbenzoic acid, p-anisic acid, 3,4,5-trimethoxybenzoic acid (eudesmic acid), trans-cinnamic acid, phthalic acid, homophthalic acid, 4-(trifluormethyl)benzoic acid, 4-iodobenzoic acid, 4-chlorobenzoic acid, 3-phenylpropionic acid, 4-(dimethylamino)benzoic acid, 4-acetamidobenzoic acid, bisphenyl-4-carboxylic acid, Methyl hydrogen terephthalate, 3-dimethylaminobenzoic acid, 4-methyl-3,5-dinitrobenzoic acid, 3-bromo benzoic acid, 4-aminobenzoic acid, 4-dimethylaminobenzoic acid, 4-(decycloxy)benzoic acid, 3-bromobenzoic acid, 1-naptholic acid, 4-dimethylaminobenzoic acid, 4-nitrobenzoic acid, terephthalic acid (and/or disodium terephthalate), 4-bromobenzoic acid, 3,5-dimethyl-p-anisic acid, 4-cycloxybenzoic acid, 4-n-pentylbenzoic acid, 4-n-heptylbanzoic acid, 4-n-hexylbenzoic acid, 4-n-butylbenzoic acid, and 2-naptholic acid.

The aromatic carboxylic acid may be selected from 4-dimethylaminobenzoic acid, 4-nitrobenzoic acid, terephthalic acid (and/or disodium terephthalate), 4-bromobenzoic acid, 3,5-dimethyl-p-anisic acid, 4-cycloxybenzoic acid, 4-n-pentylbenzoic acid, 4-n-heptylbanzoic acid, 4-n-hexylbenzoic acid, 4-n-butylbenzoic acid, and 2-naptholic acid.

The aromatic carboxylic acid may be selected from benzoic acid, toluic acid (e.g. p-toluic acid), 4-ethylbenzoic acid, 3,5-dimethylbenzoic acid, 4-t-butylbenzoic acid, p-anisic acid, 3,4,5-trimethoxybenzoic acid (eudesmic acid), trans-cinnamic acid, phthalic acid, homophthalic acid, 4-(trifluormethyl)benzoic acid, 4-iodobenzoic acid, 4-chlorobenzoic acid, 3-phenylpropionic acid, 4-(dimethylamino)benzoic acid, 4-acetamidobenzoic acid, and bisphenyl-4-carboxylic acid.

The presence and optionally identity of aromatic carboxylic acid in magnetic clusters of the invention may be confirmed using a suitable mass spectrometry method, such as analysis of the magnetic clusters using electrospray ionization mass spectrometry.

The particles may be in a population of at least 100, e.g. at least 1000. For example, for the purposes of measurement the particles may be in a population of at least 100, e.g. at least 1000. For example, in certain end use applications, the particles may conveniently be in a population of at least 100, e.g. at least 1000.

The magnetic clusters may have a mode diameter of at least about 100 nm. For example, the magnetic clusters may have a mode diameter of at least about 150 nm, at least about 200 nm, or at least about 250 nm.

The magnetic clusters may have a mode diameter of not more than about 2,500 nm. For example, the magnetic clusters may have a mode diameter of not more than about 2,000 nm, not more than about 1,500 nm, or not more than about 1,000 nm.

The magnetic clusters may have a mode diameter in the range of from about 100 nm to about 2,500 nm. The magnetic clusters have a mode diameter in the range of from about 150 nm to about 2,000 nm. The magnetic clusters have a mode diameter in the range of from about 150 nm or about 200 nm to about 1,500 nm. The magnetic clusters have a mode diameter in the range of from about 200 nm to about 1,000 nm.

The magnetic clusters may have a coefficient of variation (CV) of not more than about 20% when measured by disc centrifuge. The magnetic clusters may have a coefficient of variation (CV) of not more than about 15% when measured by disc centrifuge. The magnetic clusters may have a coefficient of variation (CV) of not more than about 10% when measured by disc centrifuge. The magnetic clusters may have a coefficient of variation (CV) of not more than about 8% when measured by disc centrifuge. The magnetic clusters may have a coefficient of variation (CV) of not more than about 5% when measured by disc centrifuge.

The magnetic clusters may have a coefficient of variation (CV) of not more than 5% when measured by disc centrifuge. The magnetic clusters may have a mode diameter in the range of from about 100 nm to about 2,500 nm and a coefficient of variation (CV) of not more than about 5% when measured by disc centrifuge.

The nanocrystals of iron oxide may comprise a mode average diameter of from about 5 nm to about 20 nm. The nanocrystals may comprise a mode average diameter of from about 10 nm to about 15 nm. The nanocrystals may comprise a mode average diameter of about 12 nm. Providing nanocrystals in the magnetic clusters in this size range may provide advantages. For example, nanocrystals in this size range may be superparamagnetic, resulting in magnetic clusters with negligible magnetic properties when not in the presence of external magnetic fields. This lack of remnant magnetism minimises clustering of the magnetic particles in the absence of external magnetic fields. In addition, the relatively high level of iron oxide in the magnetic clusters provides for efficient magnetic separation/collection of the magnetic clusters during assays.

The magnetic clusters may comprise a coating. The coating may comprise at least one layer of silica. The coating may comprise at least one layer of organic coating. The organic coating may comprise a spacer (e.g. an oligoethyleneglycol or polyethyleneglycol) and/or a polymer. Hydrophilic spacers, such as oligoethyleneglycol or polyethyleneglycol, may be particularly suitable for magnetic clusters used in biological assays, as hydrophilic spacer arms are able to interact with an aqueous environment and tend to limit non-specific protein adsorption. The coating may comprise at least one layer of silica and at least one layer of organic coating, optionally wherein the at least one layer of organic coating overcoats at least one layer of silica.

The coating may comprise functional groups, for example functional groups on spacers. The functional groups may be selected from hydroxyl groups, carboxylic acid groups, aldehyde groups, amine groups (e.g. primary or secondary aliphatic amine groups, and aromatic amine groups), thiol groups, epoxy groups, amide groups, chloromethyl groups, and tosyl-activated groups. The functional groups may be selected from a hydroxyl, a carboxylic acid (—COOH), a primary amine, a secondary amine, and an epoxy. For example, the functional groups may be selected from a carboxylic acid, a primary amine, a secondary amine, and an epoxy; e.g. the functional groups may be selected from a carboxylic acid, a primary amine, and a secondary amine.

A ligand may be attached to a functional group using methods that are known to the skilled person. Examples of such methods are provided in Chapter 14 of G. T. Hermanson, Bioconjugate Techniques, Academic Press, (3^{rd} Edition, 1996). The magnetic clusters may therefore comprise ligands, for example ligands attached to the magnetic clusters via a reacted reactive group.

The magnetic clusters may comprise at least 80% dry weight, for example at least 85% dry weight, iron oxide.

The magnetic clusters may be characterized by other features. An example of such a feature is specific surface area. The specific surface area represents the overall specific external and internal surface area of samples obtained by measuring the amount of physically adsorbed gas (typically nitrogen or argon) according to the Brunauer, Emmett and Teller (BET) method (Brunauer, Stephen; Emmett, P. H.; Teller, Edward (1938). “Adsorption of Gases in Multimolecular Layers”. Journal of the American Chemical Society. 60 (2): 309-319). The specific surface area is accordingly a function of the size of the magnetic clusters, level of porosity, and roughness of the surface. As the skilled person will appreciate, coating magnetic clusters may reduce porosity and/or surface roughness, reducing surface area compared to corresponding uncoated magnetic clusters. Exemplary magnetic clusters may have a specific surface area of from about 5 m²/g to about 100 m²/g. For example, the specific surface area may be from about 10 m²/g to about 75 m²/g.

The magnetic clusters may be obtainable by or obtained by a method disclosed herein.

### Preparation of Magnetic Clusters

The disclosure provides methods of forming magnetic clusters. The method involves providing a solution of iron (III) ions in a protic solvent and precipitating the iron out of the solution to form magnetic nanoclusters. While methods of precipitating iron (III) ions out of solution may be known in the art, the resulting magnetic particles may be highly polydisperse and/or be ferro or ferrimagnetic, both of which are undesirable for particles intended for use in assays. The inventors have determined that performing the precipitation reactions in the presence of certain aromatic carboxylic acid provides magnetic clusters that are (highly) monodisperse and paramagnetic or superparamagnetic. Such magnetic clusters are useful in assays.

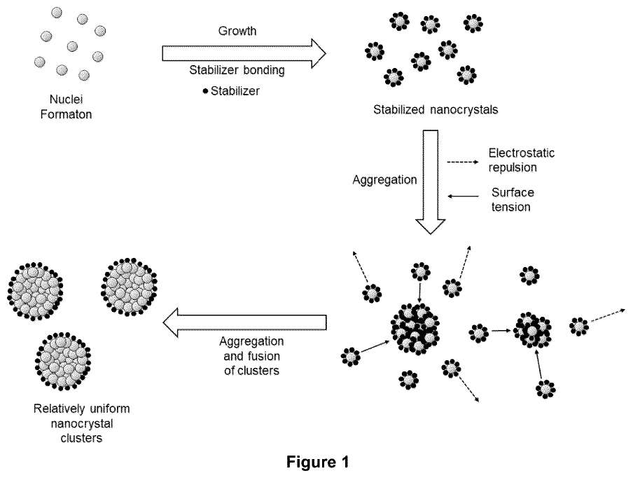

C. Cheng et al., J. Mater. Chem., 2009, 19, 8782-8788 have prepared magnetic nanoclusters by precipitating iron from solution in the presence of sodium citrate as a stabilizer. FIG. 1 (adapted from Scheme 1 of C. Cheng et al.) provides a schematic of a hypothesis for how the magnetic nanoclusters form, with an additional indication that the resulting clusters may be relatively uniform in size. For example, they determined that by changing the concentration of sodium citrate in the reaction from 0.001 M to 0.1 M, the diameter of the magnetic nanoclusters can be tuned from approximately 300 nm to 40 nm.

Without wishing to be bound by any theory, as indicated in FIG. 1, it is believed that the process of forming nanoclusters begins by the formation of iron oxide nuclei in a solution of ethylene glycol. These nuclei become iron oxide nanocrystals with citrate stabilizer adsorbed to the surface. The neighboring nanocrystals interact. Aggregation of the nanocrystals to form magnetic nanoclusters is believed to be governed by the balance between the forces of surface tension and electrostatic repulsion. The stabilizer adsorbed on the surface of the nanocrystals provides forces of electrostatic repulsion between neighboring nanocrystals. The surface tension of the nanocrystals has the opposite effect, as the nanocrystals want to aggregate to reduce surface area to volume (and hence surface energy) in ethylene glycol. Thus increasing the concentration of sodium citrate acts to increase the electrostatic repulsion as more citrate adsorbs on the surface of the nanocrystals, which has the effect of decreasing the size of the magnetic nanoclusters, as observed by Cheng et al.

We have determined that aromatic carboxylic acids, especially aromatic carboxylic acids of formula (I), provide good stabilizers of nanocrystals when preparing magnetic clusters by precipitating iron from solution. Surprisingly, unlike the situation for a citrate stabilizer, increasing the concentration of aromatic carboxylic acid stabilizer typically acts to increase the size of the magnetic nanoclusters formed. Without wishing to be bound by any theory, we consider that the aromatic carboxylic acid stabilizers do not contribute a significant electrostatic charge to the particles. Instead, the main effect of adsorbed aromatic carboxylic acid stabilizer is to increase the forces of surface tension, thus we have observed that increasing the amount of stabilizer generally results in larger magnetic clusters. Thus adjusting the amount of stabilizer allows for tuning of the size of the magnetic clusters, from the submicron to several micron scale. In addition, the presence of the aromatic rings in the stabilizer may promote a preferential orientation between stabilizer molecules (e.g. due to π-π interactions), which helps create uniform coverage of nanocrystals with the stabilizer. More uniform coverage of stabilizer also provides a more uniform distribution of electrostatic repulsion and surface tension forces between nanocrystals and clusters. This may result in magnetic clusters of a given batch with a high level of uniformity, i.e. with a low CV (e.g. a CV of less than 20% or 15%, such as a CV of less than 10%).

An embodiment provides a method of making magnetic nanoclusters. The method comprises forming a solution comprising iron (III) ions, a protic solvent, an aromatic carboxylic acid, and a precipitator; and allowing the solution to react to form a suspension comprising magnetic clusters.

The iron (III) ions may be provided as a soluble iron (III) salt. The soluble iron (III) salt may be an iron (III) halide. The soluble iron (III) salt may be iron (III) chloride.

The protic solvent may be or comprise an alcohol. The alcohol may be a diol or a polyol (such as a triol). The alcohol may be a diol, such as propylene glycol or ethylene glycol. For example, the alcohol may be ethylene glycol. The use of ethylene glycol may be preferred compared to propylene glycol, as ethylene glycol is believed to provide a solvent system that provides slower orientation and aggregations of the particles, which may promote more uniform size distribution when used in combination with aromatic carboxylic acid stabilizers in the methods of the invention. The alcohol may be a polyol, such as diethylene glycol (DEG) or tetraethylene glycol (TEG). In example methods, a diol (e.g. ethylene glycol) may be preferred to a polyol.

The presence of appreciable amounts of water (e.g. >1% water) in the protic solvent may have a negative effect on the methods of the invention, for example by affecting the surface tension and/or the uniformity of stabilizer coverage. Without wishing to be bound by any theory, it is believed that this may be due to water preferentially binding to the surface of the nanocrystals, which it is believed dampens the coordinating power of the stabilizers (see, e.g., S. W. Cao et al., New J. Chem., 2008, 32, 1526-1530; A. Kostopoulou et al., Nanoscale, 2014, 6, 3764-3776). The protic solvent may therefore comprise less than 1% water, e.g. the protic solvent may be (substantially) anhydrous.

The aromatic carboxylic acid may be a compound of formula (I):

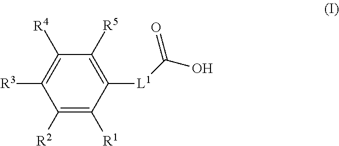

L¹ is selected from a bond, —C₁₋₆alkyl-, or —C₂₋₆alkenyl-. R¹ is selected from —H, —COOH, —(C₁₋₅alkyl)COOH, —(C₂₋₅alkenyl)COOH, or —C₁₋₄alkoxy. R² is selected from —H, -halo, —C₁₋₄alkyl, —C₁₋₄alkoxy, or —NO₂. R³ is selected from —H, -halo, —C₁₋₁₂alkyl, —C₃₋₈cycloalkly, —C₁₋₁₂alkoxy, -halo(C₁₋₁₂)alkyl, —NR⁶R⁷, —NO₂, —C(O)OH, —C(O)OR⁶, or substituted or unsubstituted phenyl. R⁴ is selected from —H, -halo, —C₁₋₄alkyl, —C₁₋₄alkoxy, or —NO₂. R⁵ is selected from —H, or —C₁₋₄alkoxy. R⁶ is selected from —H, or —C₁₋₄alkyl. R⁷ is selected from —H, —C₁₋₄alkyl, —C(O)H, —C(O)C₁₋₄alkyl. Alternatively, R₁ and R₂ together with the carbon atoms to which they are attached form a substituted or unsubstituted phenyl, and L¹, R³, R⁴, R⁵, R⁶ and R⁷ are as defined herein. In another alternative, R₂ and R₃ together with the carbon atoms to which they are attached form a substituted or unsubstituted phenyl, and L¹, R¹, R⁴, R⁵, R⁶ and R⁷ are as defined elsewhere.

In an embodiment, L¹ is selected from a bond, —C₁₋₆alkyl-, or —C₂₋₆alkenyl-. R¹ is selected from —H, —COOH, —(C₁₋₅alkyl)COOH, —(C₂₋₅alkenyl)COOH, or —C₁₋₄alkoxy. R² is selected from —H, —C₁₋₄alkyl, —C₁₋₄alkoxy. R³ is selected from —H, halo, —C₁₋₆alkyl, —C₁₋₆alkoxy, -halo(C₁₋₆)alkyl, —NR⁶R⁷, or substituted or unsubstituted phenyl. R⁴ is selected from —H, —C₁₋₄alkyl, —C₁₋₄alkoxy. R⁵ is selected from —H, or —C₁₋₄alkoxy. R⁶ is selected from —H, or —C₁₋₄alkyl. R⁷ is selected from —H, —C₁₋₄alkyl, —C(O)H, —C(O)C₁₋₄alkyl.

L¹ may be selected from a bond, —C₁₋₄alkyl-, or —C₂₋₄alkenyl-. L¹ may be a bond. L¹ may be selected from —C₁₋₄alkyl-, or —C₂₋₄alkenyl-. L¹ may be —C₁₋₆alkyl-, optionally a —C₁₋₄alkyl-. L¹ may be —C₂₋₆alkenyl-, optionally a —C₂₋₄alkenyl-.

R¹ may be selected from —H, —COOH, —(C₁₋₅alkyl)COOH, or —(C₂₋₅alkenyl)COOH. R1 may be selected from —H, or —COOH. R¹ may be —H. R¹ may be —COOH. R¹ may be —H—(C₁₋₅alkyl)COOH. R¹ may be —(C₂₋₅alkenyl)COOH. R¹ may be —C₁₋₄alkoxy.

R² may be selected from —H, —C₁₋₄alkyl, or —C₁₋₄alkoxy. R² may be selected from —H, or —C₁₋₄alkoxy. R² may be selected from —H, or —OCH₃. R² may be —H. R² may be —C₁₋₄alkyl, e.g. R² may be —CH₃. R² may be —C₁₋₄alkoxy, e.g. R² may be —OCH₃.

R₁ and R₂ together with the carbon atoms to which they are attached may form a substituted or unsubstituted phenyl.

R³ may be selected from —H, -halo, —C₁₋₆alkyl, —C₃₋₆cycloalkly, —C₁₋₆alkoxy, -halo(C₁₋₆)alkyl, —NR⁶R⁷, —NO₂, —C(O)OH, or substituted or unsubstituted phenyl. R³ may be selected from —H, -halo, —C₁₋₆alkyl, —C₁₋₆alkoxy, -halo(C₁₋₆)alkyl, —NR⁶R⁷, or substituted or unsubstituted phenyl.

R³ may be selected from —H, —C₁₋₄alkyl, -halo, —C₁₋₄alkoxy, -halo(C₁₋₄)alkyl, —NR⁶R⁷, or substituted or unsubstituted phenyl. R³ may be selected from —H, —C₁₋₄alkyl, —C₁₋₄alkoxy, -halo(C₁₋₄)alkyl, —NR⁶R⁷, or substituted or unsubstituted phenyl. R³ may be selected from —H, —C₁₋₄alkyl, —C₁₋₄alkoxy, —NR⁶R⁷, or substituted or unsubstituted phenyl. R³ may be selected from —C₁₋₄alkoxy, -halo(C₁₋₄)alkyl, —NR⁶R⁷, or substituted or unsubstituted phenyl. R³ may be selected from —C₁₋₄alkoxy, —NR⁶R⁷, or substituted or unsubstituted phenyl. R³ may be selected from —C₁₋₄alkoxy, -halo(C₁₋₄)alkyl, —NR⁶R⁷, or substituted or unsubstituted phenyl. R³ may be selected from —C₁₋₄alkoxy, —NR⁶R⁷, or unsubstituted phenyl. R³ may be selected from —H, —C₁₋₄alkyl, —Cl, —I, —OCH₃, —CF₃, —N(CH₃)₂, —NHC(O)CH₃, or phenyl. R³ may be selected from —H, —C₁₋₄alkyl, —OCH₃, —N(CH₃)₂, —NHC(O)CH₃, or phenyl. R³ may be selected from —OCH₃, —CF₃, —N(CH₃)₂, —NHC(O)CH₃, or phenyl. R³ may be selected from —OCH₃, —N(CH₃)₂, —NHC(O)CH₃, or phenyl.

R³ may be selected from —H, —C₁₋₁₀alkoxy, —NR⁶R⁷, —C(O)OR⁶, or substituted or unsubstituted phenyl. R³ may be selected from —H, —C₁₋₄alkyl, —OCH₃, —OCH₂(CH₂)₈CH₃, —N(CH₃)₂, —NHC(O)CH₃, —C(O)OCH₃, or phenyl.

R³ may be —H. R³ may be —C₁₋₆alkyl, e.g. R³ may be —C₁₋₄alkyl. R³ may be —C₁₋₆alkoxy, e.g. R³ may be —C₁₋₄alkoxy. R³ may be —OCH₃. R³ may be —NR⁶R⁷. R³ may be substituted or unsubstituted phenyl, e.g. R³ may be unsubstituted phenyl. R³ may be -halo, e.g. R³ may be —Cl. R³ may be -halo(C₁₋₆)alkyl, e.g. R³ may be -halo(C₁₋₄)alkyl. R³ may be —CF₃. R³ may be C₁₋₁₂alkyl, e.g. R³ may be —OCH₂(CH₂)₈CH₃ or —OCH₃. R³ may be —C(O)OR⁶, e.g. R³ may be —C(O)OCH₃.

R₂ and R₃ together with the carbon atoms to which they are attached may form a substituted or unsubstituted phenyl.

R⁴ may be selected from —H, —C₁₋₄alkyl, or —C₁₋₄alkoxy. R⁴ may be selected from —H, or —C₁₋₄alkoxy. R⁴ may be selected from —H, or —OCH₃. R⁴ may be —H. R⁴ may be —C₁₋₄alkyl, e.g. R⁴ may be —CH₃. R⁴ may be —C₁₋₄alkoxy, e.g. R⁴ may be —OCH₃.

R⁵ may be selected from —H, or —OCH₃. R⁵ may be —H. R⁵ may be —C₁₋₄alkoxy, e.g. R⁵ may be —OCH₃.

R⁶ may be —H or —CH₃. R⁶ may be —H. R⁶ may be —C₁₋₄alkyl, e.g. R⁶ may be —CH₃.

R⁷ may be —CH₃ or —C(O)CH₃. R⁷ may be —H. R⁷ may be —C₁₋₄alkyl, e.g. R⁷ may be —CH₃. R⁷ may be —C(O)H. R⁷ may be —C(O)C₁₋₄alkyl, e.g. R⁷ may be —C(O)CH₃.

In embodiments, not more than one of R², R³ and R⁴ may be a -halo.

The aromatic carboxylic acid may be selected from benzoic acid, toluic acid (e.g. p-toluic acid), 4-ethylbenzoic acid, 3,5-dimethylbenzoic acid, 4-t-butylbenzoic acid, p-anisic acid, 3,4,5-trimethoxybenzoic acid (eudesmic acid), trans-cinnamic acid, phthalic acid, homophthalic acid, 4-(trifluormethyl)benzoic acid, 4-iodobenzoic acid, 4-chlorobenzoic acid, 3-phenylpropionic acid, 4-(dimethylamino)benzoic acid, 4-acetamidobenzoic acid, bisphenyl-4-carboxylic acid, Methyl hydrogen terephthalate, 3-dimethylaminobenzoic acid, 4-methyl-3,5-dinitrobenzoic acid, 3-bromo benzoic acid, 4-aminobenzoic acid, 4-dimethylaminobenzoic acid, 4-(decycloxy)benzoic acid, 3-bromobenzoic acid, 1-naptholic acid, 4-dimethylaminobenzoic acid, 4-nitrobenzoic acid, terephthalic acid (and/or disodium terephthalate), 4-bromobenzoic acid, 3,5-dimethyl-p-anisic acid, 4-cycloxybenzoic acid, 4-n-pentylbenzoic acid, 4-n-heptylbanzoic acid, 4-n-hexylbenzoic acid, 4-n-butylbenzoic acid, and 2-naptholic acid.

The aromatic carboxylic acid may be selected from 4-dimethylaminobenzoic acid, 4-nitrobenzoic acid, terephthalic acid (and/or disodium terephthalate), 4-bromobenzoic acid, 3,5-dimethyl-p-anisic acid, 4-cycloxybenzoic acid, 4-n-pentylbenzoic acid, 4-n-heptylbanzoic acid, 4-n-hexylbenzoic acid, 4-n-butylbenzoic acid, and 2-naptholic acid.

The aromatic carboxylic acid may be selected from benzoic acid, toluic acid (e.g. p-toluic acid), 4-ethylbenzoic acid, 3,5-dimethylbenzoic acid, 4-t-butylbenzoic acid, p-anisic acid, 3,4,5-trimethoxybenzoic acid (eudesmic acid), trans-cinnamic acid, phthalic acid, homophthalic acid, 4-(trifluormethyl)benzoic acid, 4-iodobenzoic acid, 4-chlorobenzoic acid, 3-phenylpropionic acid, 4-(dimethylamino)benzoic acid, 4-acetamidobenzoic acid, bisphenyl-4-carboxylic acid.

The magnetic clusters formed comprise both iron oxide and the aromatic carboxylic acid. The presence and optionally identity of the aromatic carboxylic acid in magnetic clusters may be confirmed using a suitable mass spectrometry method, such as analysis of the magnetic clusters using electrospray ionization mass spectrometry.

The iron oxide of the magnetic clusters may comprise both ferrous (divalent) and ferric (trivalent) iron ions, for example the iron oxide magnetic clusters may comprise magnetite (having a composition of about Fe²⁺(Fe³⁺)₂(O²⁻)₄). Accordingly, the allowing the solution to react to form a suspension comprising magnetic clusters and/or the precipitating the iron out of the solution to form magnetic nanoclusters, may comprise reduction of some of the iron (III) ions to iron (II) ions. The reducing agent that provides the reduction may be or comprise the protic solvent (such as an alcohol, e.g. ethylene glycol). The precipitator may act as a reduction assistant; and, where the precipitator comprises an ionic species, the preceptor may be an electrostatic stabilization agent that reduces or prevents particle agglomeration.

The precipitator may be urea or an acetate, e.g. the precipitator may be an acetate. For example, the precipitator may be an acetate salt, such as an alkali metal acetate. The precipitator may be sodium acetate.

The mole ratio of the iron (III) ions to the aromatic carboxylic acid may be from about 1:0.1 to about 1:5. For example, the mole ratio of the iron (III) ions to the aromatic carboxylic acid may be from about 1:0.2 to about 1:2; e.g. the mole ratio may be from about 1:0.2 to about 1:1.5.

The mole ratio of the iron (III) ions to the precipitator may be from about 1:1 to about 1:10. For example, the mole ratio of the iron (III) ions to the aromatic carboxylic acid may be from about 1:2 to about 1:5; e.g. the mole ratio may be about 1:3.5.

The mole ratio of the iron (III) ions to the aromatic carboxylic acid and to the precipitator may be from about 1:0.1:1 to about 1:5:10. For example, the mole ratio of the iron (III) ions to the aromatic carboxylic acid and to the precipitator may be from about 1:0.2:2 to about 1:2:5; e.g. the mole ratio may be from about 1:0.2:3.5 to about 1:1.5:3.5.

The mole ratio of the iron (III) ions to the protic solvent may from about 1:30 to about 1:250. For example, the mole ratio of the iron (III) ions to the protic solvent may from about 1:30 to about 1:200; e.g. the mole ratio of the iron (III) ions to the protic solvent may from about 1:30 to about 1:150. The mole ratio of the iron (III) ions to the protic solvent may from about 1:50 to about 1:100.

The step of allowing the solution to react may comprise allowing the solution to react at an elevated temperature (e.g. of greater than about 50 °C). The elevated temperature may be a temperature of from about 150 °C to about 250 °C. For example, the elevated temperature may be a temperature of from about 170 °C to about 250 °C, e.g. a temperature of from about 180 °C to about 230 °C.

The step of allowing the solution to react may comprise allowing the solution to react at a given pressure, where the pressure represents the total (not partial) pressure. The step of allowing the solution to react may comprise allowing the solution to react at a pressure of from about 1 bar to about 15 bar. For example, the pressure may be from about 1 bar to about 10 bar, e.g. the pressure may be from about 3 bar to about 10 bar.

The exact reaction time is not critical, although it is important to allow sufficient time for the magnetic clusters to form, e.g. it may be appropriate to allow a reaction time of at least about 6 hours (e.g. at least about 8 hours). The step of allowing the solution to react may comprise allowing the solution to react for a reaction time of from about 6 h to about 36 h. For example, the reaction time may be of from about 8 h to about 24 h.

The method may further comprise separating the magnetic clusters from the suspension. The separation may be done by any suitable technique. For example decanting (optionally after centrifugation or magnetic precipitation of the magnetic clusters), filtration, etc.

The method may further comprise washing the magnetic clusters after the separating. The washing may be done with any suitable wash solution, such as an alcohol (e.g. ethanol, methanol), water, aqueous citrate (e.g. aqueous sodium citrate), acetone, (1 S,5R)-6,8-Dioxabicyclo[3.2.1]octan-4-one (Cyrene™), and the like, or a mixture thereof. The washing may comprise one or more applications of the same or different wash solutions. For instance, an exemplary step of washing the magnetic clusters may comprise washing with ethanol, then with water, then with aqueous citrate, and then with water.

The method may further comprise adding a silica coating to the separated magnetic clusters, comprising: forming a suspension comprising the separated magnetic clusters and silicates or orthosilicates; and reacting the silicates or orthosilicates to form a silica coating. A silica coating may be formed by reaction of silicates (e.g. Na₂SiO₃) at a pH of less than about 11 in the presence of the separated magnetic clusters. A silica coating may be formed by reaction of orthosilicates (e.g. tetraethyl orthosilicate) in the presence of the separated magnetic clusters. While this reaction may be performed at room temperature, in an embodiment the reaction is performed at a higher temperature, such as a temperature of at least 40 °C (e.g. a temperature of at least 60 °C or at least 90 °C).

The method may further comprise overcoating the silica coating with an organic coating. The overcoating may be performed in the presence of a water miscible solvent, such as Cyrene™ ((1S,5R)-6,8-Dioxabicyclo[3.2.1]octan-4-one). The organic coating may comprise a spacer and/or a polymer. The spacer may be or comprise an oligoethyleneglycol or a polyethyleneglycol. Hydrophilic spacers, such as oligoethyleneglycol or polyethyleneglycol, may be particularly suitable for magnetic clusters used in biological assays, as hydrophilic spacer arms are able to interact with an aqueous environment and tend to limit non-specific protein adsorption.

The coating may comprise functional groups. The functional groups may be selected from hydroxyl groups, carboxylic acid groups, aldehyde groups, amine groups (e.g. primary or secondary aliphatic amine groups, and aromatic amine groups), thiol groups, epoxy groups, amide groups, chloromethyl groups, and tosyl-activated groups. The functional groups may be selected from a hydroxyl, a carboxylic acid (—COOH), a primary amine, a secondary amine, and an epoxy. For example, the functional groups may be selected from a carboxylic acid, a primary amine, a secondary amine, and an epoxy; e.g. the functional groups may be selected from a carboxylic acid, a primary amine, and a secondary amine.

A ligand may be attached to a functional group using methods that are known to the skilled person. Examples of such methods are provided in Chapter 14 of G. T. Hermanson, Bioconjugate Techniques, Academic Press, (3^{rd} Edition, 1996). The method may comprise attaching ligands to functional groups of the magnetic clusters. For example, the method may comprise attaching ligands to functional groups of the coating of the magnetic clusters.

The magnetic clusters may have a mode diameter of at least about 100 nm. For example, the magnetic clusters may have a mode diameter of at least about 150 nm, at least about 200 nm, or at least about 250 nm.

The magnetic clusters may have a mode diameter of not more than about 2,500 nm. For example, the magnetic clusters may have a mode diameter of not more than about 2,000 nm, not more than about 1,500 nm, or not more than about 1,000 nm.

The magnetic clusters may have a mode diameter in the range of from about 100 nm to about 2,500 nm. The magnetic clusters have a mode diameter in the range of from about 150 nm to about 2,000 nm. The magnetic clusters have a mode diameter in the range of from about 150 nm or about 200 nm to about 1,500 nm. The magnetic clusters have a mode diameter in the range of from about 200 nm to about 1,000 nm.

The magnetic clusters may be monodisperse.

The magnetic clusters may have a coefficient of variation (CV) of not more than about 20% when measured by disc centrifuge. The magnetic clusters may have a coefficient of variation (CV) of not more than about 15% when measured by disc centrifuge. The magnetic clusters may have a coefficient of variation (CV) of not more than about 10% when measured by disc centrifuge. The magnetic clusters may have a coefficient of variation (CV) of not more than about 8% when measured by disc centrifuge. The magnetic clusters may have a coefficient of variation (CV) of not more than about 5% when measured by disc centrifuge.

The magnetic clusters may have a coefficient of variation (CV) of not more than 5% when measured by disc centrifuge. The magnetic clusters may have a mode diameter in the range of from about 100 nm to about 2,500 nm and a coefficient of variation (CV) of not more than about 5% when measured by disc centrifuge.

The nanocrystals of iron oxide may comprise a mode average diameter of from about 5 nm to about 20 nm. The nanocrystals may comprise a mode average diameter of from about 10 nm to about 15 nm. The nanocrystals may comprise a mode average diameter of about 12 nm. Providing nanocrystals in the magnetic clusters in this size range may provide advantages. For example, nanocrystals in this size range may be superparamagnetic, resulting in magnetic clusters with negligible magnetic properties when not in the presence of external magnetic fields. This lack of remnant magnetism minimises clustering of the magnetic particles in the absence of external magnetic fields. In addition, the relatively high level of iron oxide in the magnetic clusters provides for efficient magnetic separation/collection of the magnetic clusters during assays.

The magnetic clusters may comprise at least 80% dry weight, for example at least 85% dry weight, iron oxide.

### Uses of Magnetic Clusters

The magnetic clusters can be used in many applications, e.g. polynucleotide sequencing, bioassays, information storage, colour imaging, in vitro diagnostics, bioprocessing, diagnostic microbiology, biosensors and drug delivery. The magnetic clusters are particularly useful in assays, (e.g. where the polymer particles are conjugated with a target analyte, such as an affinity ligand), as the magnetic material allows the particles (and anything bound thereto) to be separated from bulk solution by application of a magnetic field.

In particular, coated magnetic clusters of the invention (e.g. comprising silica and/or functional groups) can be used as a reagent for isolating and/or detecting biomaterials, particularly, nucleic acids, and can isolate and purify nucleic acid in a high yield. In addition, the highly active silica magnetic nanoparticles can also be used for protein purification, antibody purification, enzyme immunoassay, peptide purification, endotoxin removal and the like.

Nucleic acid purification and manipulation are essential processes of all molecular biology laboratories. Nucleic acids can be isolated from a wide variety of sources, including samples containing viruses, bacteria, plant or animal or human cells or tissues. Nucleic acids may also be derived from cell-free sources, clinical samples such as blood, plasma, serum, swabs, biopsies, tissues, various environmental sources, or from in vitro reactions. Accordingly, nucleic acids are commonly used as markers for the detection of biological entities, e.g. viruses, bacteria, and may therefore be used in the diagnosis of various diseases. The method is also suitable for the isolation of free circulating nucleic acids such as tumour DNA and RNA as well as fetal DNA from plasma and/or serum.

In certain cases the sample can be used without pretreatment. However, nucleic acids typically must be isolated from their environment and amplified before they can be efficiently detected or further manipulated. Thus, in many cases the sample must first be digested by a suitable method and the biological material to be contained in the sample are released. Methods for disrupting samples and cells are well known in the art and can be chemical, enzymatic or physical in nature. Also a combination of these methods is possible.

In this case there may be various factors found to be advantageous for different biological materials. In many instances the sample is contacted with a lysis reagent including for example ionic and non-ionic detergents, such as e.g. SDS, lids or sarkosyl in appropriate buffers, chaotropic salts, such as for example guanidine hydrochloride (GHCL), guanidinium thiocyanate (GTC), guanidinium isothiocyanate (GITC), sodium iodide, sodium perchlorate inter alia. Also mechanical tearing apart, such as for example by means of a French Press, ultrasound, milling with glass beads, nickel beads, aluminum or in liquid nitrogen may be performed in some instances. Other treatments such as enzymatic lysis, for example with lysozyme, proteinases, Proteinase K or cellulases or by means of other commercially available enzymes for lysis; lysis of the cells by bacteriophage or virus infections; freeze-drying; osmotic shock; microwave treatment; heat treatment; for example by heating or boiling or freezing, e.g. in dry ice or liquid nitrogen and thawing; alkaline lysis are possible.

In one example, silica coated magnetic clusters of the invention may be used to isolate nucleic acids, by adapting a widely known method of isolating nucleic acid onto a silica surface using a chaotropic reagent (R. Boom et al., J. Clin. Microbiol., Vol 28(3), p 495-503 (1990)). In this method, when magnetic clusters coated with silica bind to nucleic acid by the chaotropic reagent, and the nucleic acid is isolated by separating the silica magnetic particles using an external magnetic force.

In the first step of this exemplary method, the silica coated magnetic clusters of the present invention are added to a sample containing the nucleic acid to be isolated to induce the binding of the nucleic acid to the silica magnetic nanoparticles.

Herein, a binding buffer is used. As the binding buffer, a chaotropic reagent may be used. Chaotropic reagents include guanidine salt, urea, chloride, iodide, perchlorate, (iso)thiocyanate and the like, and specific examples include, but are not limited to, sodium perchlorate, guanidine hydrochloride, guanidine isothiocyanate, potassium iodide, potassium thiocyanate, sodium chloride, sodium isothiocyanate, magnesium chloride, sodium iodide, etc. The chaotropic reagent is preferably used at a concentration of 1-8 mol/L.

Alternatively or in addition, a binding buffer may comprise a branched or unbranched alcohol such as e.g. ethanol or isopropanol. WO 91/12079 describes a method whereby nucleic acid is trapped on the surface of a solid phase by precipitation. Generally speaking, alcohols and salts are used as precipitants. Depending on the size of the nucleic acids to be isolated, the alcohol may be present during the binding step at a concentration of more than 5% (v/v) and less than 60% (v/v), preferably, less than 40% (v/v). In some instances, the alcohol may be present during the binding step at a concentration between about 10% (v/v) and about 30% (v/v).

U.S. Pat. Nos. 5,705,628 and 5,898,071 describe methods of isolating nucleic acid fragments using a combination of large molecular weight polyalkylene glycols (e.g. polyethylene glycols) at concentrations of from 7 to 13% with salt in the range of 0.5 to 5M to achieve binding to functional groups on a solid support which acts as a bioaffinity absorbent for DNA. In other examples tetraethylene glycol may be used to precipitate nucleic acid on a solid phase as described in U.S. Pat. No. 8,569,477. Any of these known methods may be used for binding of nucleic acids to the silica-coated particles of the invention.

The second step of the method for nucleic acid isolation is a step of isolating the silica coated magnetic clusters having the nucleic acid bound thereto. In this step, the silica coated magnetic clusters having the nucleic acid bound thereto are collected on the wall of the container by an external magnetic force, and unbound material is separated, followed by washing.

The third step is a step of removing the external magnetic force and isolating the nucleic acid from the silica magnetic nanoparticles having the nucleic acid bound thereto. In this step, the nucleic acid bound to the silica magnetic particles is isolated using an elution buffer (such as a tris-(hydroxymethyl)amino methane buffer).

Thus, the silica-coated particles of the invention can be used for generic capture of nucleic acids. Alternatively, specific capture of nucleic acids can be achieved by hybridization to an oligonucleotide immobilized on the particle. Isolated nucleic acids can be used for further manipulations or detected by an amplification technology.

An embodiment provides use of magnetic clusters of the invention in an assay such as a bioassay. The magnetic clusters are particularly suitable for various types of assays due to the following advantageous properties: 1) the small size of the nanoparticles ensures a large surface area available for target capture; 2) a high number of particles per sample volume ensures fast and efficient capture of the target; 3) The magnetic properties described herein allow for easy automation of the assay/isolation protocol, and 4) the high iron content ensures efficient magnetic separation in viscous samples.

The magnetic clusters used in the assay may comprise a coating. The coating may comprise at least one layer of silica. The coating may comprise at least one layer of organic coating. The organic coating may comprise a spacer (e.g. an oligoethyleneglycol or polyethyleneglycol) and/or a polymer. Hydrophilic spacers, such as oligoethyleneglycol or polyethyleneglycol, may be particularly suitable for magnetic clusters used in biological assays, as hydrophilic spacer arms are able to interact with an aqueous environment and tend to limit non-specific protein adsorption. The coating may comprise at least one layer of silica and at least one layer of organic coating, optionally wherein the at least one layer of organic coating overcoats at least one layer of silica.

The coating may comprise functional groups, for example functional groups on spacers. The functional groups may be selected from hydroxyl groups, carboxylic acid groups, aldehyde groups, amine groups (e.g. primary or secondary aliphatic amine groups, and aromatic amine groups), thiol groups, epoxy groups, amide groups, chloromethyl groups, and tosyl-activated groups. The functional groups may be selected from a hydroxyl, a carboxylic acid (—COOH), a primary amine, a secondary amine, and an epoxy. For example, the functional groups may be selected from a carboxylic acid, a primary amine, a secondary amine, and an epoxy; e.g. the functional groups may be selected from a carboxylic acid, a primary amine, and a secondary amine.

A ligand may be attached to a functional group using methods that are known to the skilled person. Examples of such methods are provided in Chapter 14 of G. T. Hermanson, Bioconjugate Techniques, Academic Press, (3^{rd} Edition, 2013). The magnetic clusters used in the assay may therefore comprise ligands, for example ligands attached to the magnetic clusters via a reacted reactive group.

In an embodiment, the magnetic clusters comprise a (surface) capture ligand. The capture ligand may be selected from the group consisting of antibodies, antibody fragments, peptides, carbohydrates, haptens, aptamers, and oligonucleotides.

The magnetic clusters may comprise surface functionalization for capture of a class of molecules. For example, the surface functionalization may comprise ionic groups or hydrophobic groups.

The assay may comprise a process selected from the group consisting of immunoprecipitation, nucleic acid capture, immunoassay, and/or lateral flow assay.

For example, immunoprecipitation may be performed direct or indirect (typically with Protein A or G, Streptavidin or a secondary antibody first bound to the particle) binding of an antibody for capture of a class of proteins or protein complexes. The captured proteins may be analyzed by mass spectrometry, gel electrophoresis or protein array technologies.

The magnetic nanoparticles may also be used in immunoassays using a direct or indirect binding of an antibody or antigen and detecting the analyte with a sandwich immunoassay or competitive immunoassay as known in the art. The detection system may comprise chemiluminescent, electrochemiluminescent, colorimetric or electrochemical detection means.

Further, the magnetic particles may be used as a reporter in an immunoassay by binding a detection antibody to the particle. The particle may then be used in an assay where the capture antibody is immobilized on a sensor surface capable of detecting a change in a magnetic field.

Due to the small size particles of the invention may also be used in lateral flow assays where the particle would act as the reporter through colorimetric detection, detection using a magnetism-based sensor, or the like.

The assay may comprise a process selected from the group consisting of protein purification, antibody purification, enzyme immunoassay, peptide purification, and endotoxin removal.

An embodiment provides a method for isolating nucleic acids from a sample containing biological material comprising:
(a) contacting the sample containing biological material with a lysis solution;
(b) contacting the sample from (a) with silica-coated magnetic clusters (e.g. as defined herein) under conditions suitable to adsorb nucleic acids in the sample to the silica-coated magnetic clusters;
(c) washing the silica-coated magnetic particles from (b); and
(d) desorbing (eluting) the nucleic acids from the silica-coated magnetic particles.

An exemplary lysis solution comprises:
(a) a buffer (e.g. Tris-HCl);
(b) a chelating agent (e.g. EDTA);
(c) a chaotropic agent (e.g. a guanidinium salt, such as guanidinium isothiocyanate);
(d) a detergent (e.g. 2-[4-(2,4,4-trimethylpentan-2-yl)phenoxy]ethanol (Triton X-100) or sodium lauroyl sarcosinate (sarkosyl)); and optionally
(e) a reducing agent (e.g. tris(2-carboxyethyl)phosphine (TCEP)); and/or
(f) a nucleic acid carrier (e.g. a polymer, such as glycogen).

An embodiment provides an in vitro method of detecting a nucleic acid from an infectious agent in a biological sample (e.g. an oral and/or nasal sample) comprising:
(a) isolating nucleic acids from a biological sample suspected of containing an infectious agent (e.g. a virus, such as a coronavirus) with magnetic clusters of the invention using a method disclosed herein; and
(b) analysing the nucleic acids from (a) for the presence of a nucleic acid from the infectious agent.

Another embodiment provides an in vitro method of determining whether a subject is infected with an infectious agent (e.g. a virus, such as a coronavirus) comprising:
(a) isolating nucleic acids from a biological sample (e.g. an oral and/or nasal sample) from a subject suspected of having an infection with magnetic clusters of the invention using a method disclosed herein; and
(b) analysing the nucleic acids from (a) for the presence of a nucleic acid from the infectious agent (e.g. virus, such as a coronavirus), wherein detection of a nucleic acid from the infectious agent indicates that the subject has an infection.

Described particles and methods may be used to isolate various types of nucleic acids such as DNA or RNA, whether obtained from a biological sample or by in vitro or de novo synthesis. Magnetic clusters of the invention are particularly suitable for isolation or purification or enrichment of gDNA, cell free nucleic acids (cfDNA, cfRNA), total RNA, mRNA, microRNAs (miRNAs), long non-coding RNAs (lncRNAs) or circular RNAs (circRNAs).

The nucleic acids obtained from the methods may be further processed (e.g. by PCR) or analysed, e.g. to detect an infectious agent as described above. The step of analysing the nucleic acids may use any nucleic acid analysis. For instance, the nucleic acids may be analysed by mass spectrometry or to determine their sequence (e.g. by nucleic acid sequencing), although actual sequence determination may not be required—any method of analysing the sequence may be used.

Particles described herein may also be used to isolate, purify, concentrate or deplete various different targets including cells, organelles, exosomes, microvesicles, apoptotic bodies, liposomes, virus, bacteria, parasites, plant cells etc. from a given sample. Various types of samples may be used depending on the target. For example, a sample may originate from various sources such as blood (whole blood, plasma, serum), bone marrow, tissue, feces, urine, tears, saliva, cell culture, perilymph or cerebrospinal fluid, milk water, sewage etc. Further, a biological sample may be obtained from different species such as microbes, viruses, plants, mammals, or human.

In some instances, the target to be isolated, purified, concentrated or depleted from a sample may be a cell or cells. In some instances, the cells may be cancer cells, CTC, stem cells, neuron, immune cells, endothelial cells, epithelial cells, or organ specific cells (beta-cells, hepatocytes, glia cells). In some examples the immune cells such as T cells, B-cells or natural killer cells.

In some embodiments particles of the invention may be used in a magnetic particle processing system for use with biological cells as described in WO2022/081519 A1. For example, such system may be used to attach magnetic clusters to biological cells in a sample and/or separate the magnetic beads with attached cells from the sample.

In some examples, the magnetic clusters may be coated with antibodies having binding affinity for a protein selected from the group consisting of CD3, CD4, CD5, CD6, CD8, CD25, CD27, CD28, CD137, and CD278 or for a protein comprising CD3 or CD28.

In some embodiments cells to be isolated or depleted from a sample may be T cells wherein the cells are attached to the magnetic particles through an antigen-antibody interaction. The cells may then be collected or removed from the sample (e.g. by magnetic force) and subsequently detached from the magnetic particles by disruption of the antigen-antibody interaction (e.g. by cleavage of the antibody and separation of the antibody from the particles).

In some embodiments the antibody may be linked to the magnetic particles by a ligand. Bound cells may then be detached from the magnetic particles by disruption of the ligand interaction with the antibody or the magnetic particle.

Cells isolated by using particles of the invention may be used for multiple downstream assays or applications such as single cell analysis (scRNAseq) or CAR-T cell based applications.

In other embodiments magnetic clusters described herein may be used in mRNA in vitro transcription and/or purification methods. For example, a first type of magnetic clusters (e.g. functionalized with streptavidin) may be used to immobilize a template (e.g. a biotinylated DNA template) for in vitro transcription. A second type of magnetic clusters (e.g. coated with carboxylic acid) may then be used to capture or purify in vitro transcribed mRNA. Such uses are described e.g. in WO 2022/101304 A1.

In some examples a method for producing purified mRNA molecules may comprise: (a) fixing a first magnetic particle in place by a magnetic field, wherein an in vitro transcription (IVT) template is linked to the first magnetic particle; (b) contacting the first magnetic particle of step (a) with a reagent mixture suitable for IVT of the template under condition in which IVT occurs, thereby producing an mRNA molecule, and (c) separating the mRNA molecule from the first magnetic particle, thereby producing the purified mRNA molecule. The method may further comprise: (d) contacting the purified RNA molecule of step (c) with a second magnetic particle under conditions that allows for the purified mRNA molecule to remain associated with the second magnetic bead during washing, (e) washing of the second magnetic bead while the second magnet bead is fixed in place by a magnetic field, and (f) releasing the purified RNA molecule from association with the second magnetic bead, thereby producing a highly purified RNA molecule. In some instances, the IVT template may be produced by polymerase chain reaction (PCR) optionally wherein one or more biotinylated primer is used in the PCR and results in the formation of a biotinylated IVT template. The biotinylated IVT template may be attached to the magnetic particle through an interaction between the biotin of the biotinylated IVT template and a group on the magnetic particle with affinity for biotin. Further, in many instances the IVT template may comprise an open reading frame encoding a protein and a promoter operably connected to the open reading frame. In some embodiments free carboxylic acid groups may be present on the surface of the second magnetic particle. The purified mRNA molecule may encode a protein or pathogen such as e.g. a viral protein. RNA molecules produced and/or purified using particles of the invention may be used in various types of applications such as vaccine compositions.

In further embodiments magnetic clusters described herein may be used to isolate organelles, vesicles, virions, virus-like particles or exosomes from a sample based on the attachment of antibody- or ligand-functionalized particles to surface markers. Such target may be derived from living organisms or may have been grown in/derived from cell cultures.

For example, surface markers which may be used for isolation, purification or fractionation of exosomes or microvesicles include but are not limited to tumour markers and MHC class II markers. MHC class II markers which have been associated with exosomes include HLA DP, DQ and DR haplotypes. Other surface markers associated with exosomes include CD9, CD81, CD63 and CD82 (Thery et al. Nat. Rev. Immunol. 2 (2002) 569-579; Valadi et al. Nat. Cell. Biol. 9 (2007) 654-659. Vesicles (e.g., exosomes) generated by T cells may be separated from other biological materials using magnetic particles that form an affinity linkage with CD3, CD4 and/or CD8 receptors. Antibody-coated magnetic clusters may be added to an exosome containing sample and incubated at 2-8 °C or at room temperature from 5 minutes to overnight. Particles with bound exosomes may then be collected using a magnet. The isolated, particle-bound exosomes may then be resuspended in an appropriate buffer such as phosphate buffered saline and used for downstream analysis (qRT-PCR, sequencing, Westerns, flow cytometry etc.). Similar protocols may be used for any other surface marker on exosomes or other vesicles for which an antibody or other specific ligand is available. Indirect binding methods such as those using biotin-avidin may also be used. Once an isolated exosome sample has been prepared, the contents of the exosome may be extracted for study and characterization. Biological material which may be extracted from exosomes includes proteins, peptides, RNA and DNA, lipids. For example, the mirVana™ PARIS Kit (AM1556, Life Technologies) may be used to recover native protein and RNA species, including small RNAs such as miRNA, snRNA, and snoRNA, from exosomes.

In further embodiments magnetic clusters described herein may be used for isolation of peptides, proteins or other macromolecules.

Proteins purified by methods using magnetic clusters may also be bound to affinity reagents that bind to the protein either to a naturally occurring region of the protein or to an exogenously added tag. In addition to examples such as Protein A binding to antibodies, such methods include those where an antibody with specificity for the protein being purified is linked to a particle. For example, when the protein is an antibody (e.g., an IgG, an IgA, an IgD, an IgM, an IgE, etc.) or a mixture of antibodies (e.g., IgG antibodies present in serum, etc.), the ligand may be Protein A, Protein G, Protein L, or one or more functional variant of one or more of these proteins.

Thus, proteins purification methods may also be based on association with an exogenously added affinity tag. By this it is meant that the affinity tag is not normally present in the naturally occurring protein. Exemplary tags and binding partners that may be present in compositions and used in methods set out herein include maltose-binding protein (MBP)/amylose, and the glutathione-S— transferase (GST)/glutathione tags, polyhistidine (His)/metal ions (e.g., copper and cobalt), streptavidin/biotin (e.g., N-ethyl-biotin), and antigen-antibody reactions (epitope) tags (e.g., c-Myc tag/anti-c-Myc antibody, FLAG/anti-FLAG antibody, and hemagglutinin (HA) tag/anti-HA antibody. Methods in protein purification workflows, as well as other workflows, may vary widely but, in some instances, magnetic particles described herein may be held in place by a magnetic field and then contacted with a protein binding partner or alternatively may be contacted with a protein binding partner and then held in place by a magnetic field. In both such instances, the supports may be held in place and washed. It may be desirable to release a protein from the particle, for example, after a process by which the protein has been separated from other materials (e.g., cell debris). The process by which the protein is released from the particle will be determined by the protein and/or the nature of the association between the protein and the support. Protein release from magnetic particle may be mediated by the use of protease cleavage either within the protein or between the protein and an exogenously added tag. Exemplary proteases that may be used include, a rhinovirus 3C protease, a TVMV protease, a plum pox virus protease, turnip mosaic virus protease, tobacco etch virus (TEV) protease, thrombin, Factor Xa, and enteropeptidase.

One type of protein purification method that allows for both purification and release of the protein from supports uses biotin and biotin derivative (e.g., biotin, desthiobiotin, N-ethyl-biotin) in conjunction with biotin binding proteins. Suitable biotin derivatives are set out e.g. in U.S. Pat. No. 9,567,346.

In some instances, an antibody or other protein with ligand binding activity is associated with a magnetic particle through a low affinity biotin derivative (e.g., desthiobiotin, N-ethyl-biotin, etc.)/biotin binding protein (e.g., avidin, streptavidin, neutravidin, etc.) association. Release of the low affinity biotin derivative from the biotin binding protein is mediated by competition with high affinity biotin (e.g., d-biotin).

Cell capture and release methods that may be used for protein purification, as well as for cells, viruses, virus like particles and other biological molecules, are those that use the CAPTURESELECT™ N-Ethyl Biotin (NEB) Anti-CD4 Conjugate (Thermo Fisher Scientific, cat. no. 7113762100) and CAPTURESELECT™ N-Ethyl Biotin (NEB) Anti-CD8 Conjugate (Thermo Fisher Scientific, cat. no. 7113772100). In this instance, a biotinylated anti-CD4 antibody or anti-CD8 antibody is bound to a magnetic particle through a biotin/streptavidin association. In summary, methods using magnetic clusters of the invention may be set out as follows: First, anti-CD4 antibodies or anti-CD8 antibodies biotinylated with NEB are contacted with streptavidin coated magnetic particles. The particles are then contacted with CD4+ or CD8+ T cells under conditions that allows for binding of the T cells through an NEB/streptavidin association. The particles are then washed after a short incubation. In many instances, the particles will be held in place by a magnetic field during the washings. After washing, the particles are contacted with a release reagent containing d-biotin. The supernatant with the CD4+ or CD8+ T cells is then removed. In many instances, the particles will be held in place by a magnetic field during the washings. The result being a purified population of CD4+T or CD8+ T cells with few or no particles present.

Viruses and virus like particles may be isolated by association with supports (e.g., magnetic beads). The manner by which viruses and virus like particles associate with supports may vary with the structures of the individual viruses and virus like particles. By way of example, adeno-associated virus (AAV) is non-enveloped and antibodies have been developed with binding affinity to AAV capsid proteins. Further, AAV capsid variations (serotypes) are known and result in different AAV serotype having different cell and tissue specificities.

Enveloped viruses and virus like particles may also be purified using particles provided herein. Enveloped viruses and virus like particles, as well as cells and exosomes, may be purified, for example, through the use of affinity agents. As part of such methods, enveloped viruses and virus like particles many be purified based upon association of a ligand with a protein present in the envelop of the virus and virus like particles that one seeks to purify. An example of such a method is set out in Mekkaoui et al., “Lentiviral Vector Purification Using Genetically Encoded Biotin Mimic in Packaging Cell”, Mol. Ther. Methods Clin. Dev., 11:155-165 (2018).

In further embodiments, magnetic clusters described herein may be used for selectively removing small molecules, analytes cations, anions, ions or environmental toxins (such as selenium) from drainage waters or other samples. For such uses the magnetic clusters may be functionalized with certain coatings as described above. For example, Dextran, sugars, PEG, PEG-OH, other modified PEG moieties, polyvinyl alcohol, gold, azide, carboxyl groups, activated carbon, Zeolites, amine, polyacrylic acid, charged polymers, or others may be used as surface functionalization.

### Kits

Magnetic clusters of the invention may be provided as part of a kit. For example, where the magnetic clusters of the invention are intended for use in an assay, it may be convenient to provide a kit comprising the magnetic clusters.

Accordingly, an embodiment of the invention provides a kit comprising magnetic clusters of the invention.

The kit may comprise one or more buffers. The buffers may comprise a lysis buffer, and/or a binding buffer, and/or a washing buffer and/or an elution buffer. As the skilled person will appreciate, the specific composition of a given buffer will depend on the assay and target analyte(s). For example, where the assay comprises isolation of nucleic acid, the buffers may comprise a lysis buffer (e.g. an exemplary lysis solution described herein), and/or a nucleic acid binding buffer, and/or a washing buffer, and/or an elution buffer.

The kit may comprise a magnet for isolating the magnetic clusters.

The kit may also comprise an instruction manual. The “instruction manual” is a printed matter describing how to use the kit, for instance, the method of preparing reagents, recommended preparation conditions, and the like. The instruction manual includes those appearing on labels attached to the kit, packages housing the kit, and the like, as well as handling brochures in a pamphlet or leaflet form. In addition, the instruction manual includes information that is disclosed or provided via an electronic medium such as the internet.

A kit comprising components for use in a method of isolating nucleic acids from a biological sample, e.g. lysis buffer and silica-coated magnetic particles, is also provided.

### FURTHER EMBODIMENTS

The disclosure includes the embodiments of the following numbered clauses:
1. Magnetic clusters, wherein the magnetic clusters comprise nanocrystals of iron oxide and an aromatic carboxylic acid.
2. The magnetic clusters of clause 1, wherein the aromatic carboxylic acid is a compound of formula (I):

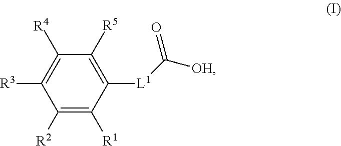

wherein:
L¹ is selected from a bond, —C₁₋₆alkyl-, or —C₂₋₆alkenyl-;
R¹ is selected from —H, —COOH, —(C₁₋₅alkyl)COOH, —(C₂₋₅alkenyl)COOH, or —C₁₋₄alkoxy;
R² is selected from —H, —C₁₋₄alkyl, —C₁₋₄alkoxy;
R³ is selected from —H, -halo, —C₁₋₆alkyl, —C₁₋₆alkoxy, -halo(C₁₋₆)alkyl, —NR⁶R⁷, or substituted or unsubstituted phenyl;
R⁴ is selected from —H, —C₁₋₄alkyl, —C₁₋₄alkoxy;
R⁵ is selected from —H, or —C₁₋₄alkoxy;
R⁶ is selected from —H, or —C₁₋₄alkyl; and
R⁷ is selected from —H, —C₁₋₄alkyl, —C(O)H, —C(O)C₁₋₄alkyl.
3. The magnetic clusters of clause 2, wherein L¹ is selected from a bond, —C₁₋₄alkyl-, or —C₂-4alkenyl-.
4. The magnetic clusters of clause 2 or clause 3, wherein L¹ is a bond.
5. The magnetic clusters of clause 2, wherein L¹ is selected from —C₁₋₄alkyl, or —C₂₋₄alkenyl; optionally wherein L¹ is a —C₂₋₄alkenyl-.
6. The magnetic clusters of any of clauses 2 to 5, wherein R¹ is selected from —H, —COOH, —(C₁₋₅alkyl)COOH, or —(C₂₋₅alkenyl)COOH.
7. The magnetic clusters of any of clauses 2 to 6, wherein R¹ is selected from —H, or —COOH.
8. The magnetic clusters of any of clauses 2 to 7, wherein R² is selected from —H, or —C₁₋₄alkoxy.
9. The magnetic clusters of any of clauses 2 to 8, wherein R² is selected from —H, or —OCH₃.
10. The magnetic clusters of any of clauses 2 to 9, wherein R³ is selected from —H, —C₁₋₄alkyl, —C₁₋₄alkoxy, —NR⁶R⁷, or substituted or unsubstituted phenyl;
optionally wherein R³ is selected from —C₁₋₄alkoxy, —NR⁶R⁷, or substituted or unsubstituted phenyl.
11. The magnetic clusters of any of clauses 2 to 10, wherein R⁶ is —H or —CH₃, and/or wherein R⁷ is —CH₃ or —C(O)CH₃.
12. The magnetic clusters of any of clauses 2 to 11, wherein R³ is selected from —H, —C₁₋₄ alkyl, —OCH₃, —N(CH₃)₂, —NHC(O)CH₃, or phenyl;
optionally wherein R³ is selected from —OCH₃, —N(CH₃)₂, —NHC(O)CH₃, or phenyl.
13. The magnetic clusters of any of clauses 2 to 12, wherein R⁴ is selected from —H, or —C₁. 4alkoxy.
14. The magnetic clusters of any of clauses 2 to 13, wherein R⁴ is selected from —H, or —OCH₃.
15. The magnetic clusters of any of clauses 2 to 14, wherein R⁵ is —H.
16. The magnetic clusters of any of clauses 2 to 16, wherein R¹ and R⁵ are both —H.
17. The magnetic clusters of any of clauses 2 to 16, wherein R³ is —OH, R² is —C₁₋₄alkoxy, and R³ is —C₁₋₄alkoxy.
18. The magnetic clusters of any preceding clause, wherein the aromatic carboxylic acid is selected from benzoic acid, toluic acid (e.g. p-toluic acid), 4-ethylbenzoic acid, 3,5-dimethylbenzoic acid, 4-t-butylbenzoic acid, p-anisic acid, 3,4,5-trimethoxybenzoic acid (eudesmic acid), trans-cinnamic acid, phthalic acid, homophthalic acid, 3-phenylpropionic acid, 4-(dimethylamino)benzoic acid, 4-acetamidobenzoic acid, bisphenyl-4-carboxylic acid.
19. The magnetic clusters of any proceeding clause, wherein the magnetic clusters are monodisperse.
20. The magnetic clusters of any preceding clause, wherein the magnetic clusters have a mode diameter in the range of from about 100 nm to about 2,500 nm;
optionally wherein the magnetic clusters have a mode diameter in the range of from about 200 nm to about 1,000 nm.
21. Monodisperse magnetic clusters comprising nanocrystals of iron oxide, wherein the magnetic clusters have a mode diameter in the range of from about 100 nm to about 2,500 nm and a coefficient of variation (CV) of not more than 5% when measured by disc centrifuge.
22. The monodisperse magnetic clusters of clause 21, wherein the magnetic clusters further comprise an aromatic carboxylic acid, optionally wherein the aromatic carboxylic acid is as defined in any of clauses 2 to 18.
23. The magnetic clusters of any of clauses 1 to 19, or monodisperse magnetic clusters of clause 21 or clause 22, wherein the nanocrystals of iron oxide comprise a mode average diameter of from about 5 nm to about 20 nm, optionally wherein the nanocrystals have a mode average diameter of from about 10 nm to about 15 nm, further optionally wherein the nanocrystals have a mode average diameter of about 12 nm.
24. The magnetic clusters of any of clauses 1 to 19 or 23, or monodisperse magnetic clusters of any of clauses 21 to 23, further comprising a coating.
25. The magnetic clusters or monodisperse magnetic clusters of clause 24, wherein the coating comprises at least one layer of silica.
26. The magnetic clusters or monodisperse magnetic clusters of clause 23 or clause 24, wherein the coating comprises at least one layer of organic coating, optionally wherein the at least one layer of organic coating overcoats at least one layer of silica.
27. The magnetic clusters or monodisperse magnetic clusters of any of clauses 23 to 26, wherein the coating comprises functional groups;
   optionally wherein the functional groups are selected from hydroxyl groups, carboxylic acid groups, aldehyde groups, amine groups, thiol groups, epoxy groups, amide groups, chloromethyl groups, and tosyl-activated groups;
   further optionally wherein the functional groups are selected from a hydroxyl, a carboxylic acid (—COOH), a primary amine, a secondary amine, or epoxy; further optionally wherein the functional groups are selected from a carboxylic acid (—COOH), a primary amine, a secondary amine, or an epoxy.
28. A method of making magnetic clusters, comprising:
   forming a solution comprising iron (III) ions, a protic solvent, an aromatic carboxylic acid, and a precipitator; and
   allowing the solution to react to form a suspension comprising magnetic clusters.
29. The method of clause 28, wherein the iron (III) ions are provided as a soluble iron (III) salt, optionally wherein the soluble iron (III) salt is an iron (III) halide, such as an iron (III) chloride salt.
30. The method of clause 28 or clause 29, wherein the protic solvent is alcohol, optionally a diol or polyol, further optionally wherein the alcohol is ethylene glycol.
31. The method of any of clauses 28 to 30, wherein the aromatic carboxylic acid is as defined in any of clauses 2 to 18.
32. The method of any of clauses 28 to 31, wherein the precipitator is an acetate, optionally sodium acetate.
33. The method of any of clauses 28 to 32, wherein allowing the solution to react comprises one or more of the following conditions:
   (a) an elevated temperature, optionally of from 150 °C to 250 °C; and/or
   (b) a pressure of from about 1 bar to about 15 bar; and/or
   (c) a reaction time of from about 6 h to about 36 h.
34. The method of any of clauses 28 to 33, further comprising separating the magnetic clusters from the suspension.
35. The method of clause 34, further comprising washing the magnetic clusters.
36. The method of clause 34 or clause 35, further comprising adding a silica coating to the separated magnetic clusters, comprising:
   forming a suspension comprising the separated magnetic clusters and silicates or orthosilicates; and
   reacting the silicates or orthosilicates to form a silica coating.
37. The method of clause 36, wherein reacting the silicates to form a silica coating comprises lowering the pH of the suspension to less than 11.
38. The method of clause 36, wherein reacting the orthosilicates to form a silica coating comprises raising the temperature of the suspension to in excess of 40 °C, optionally to in excess of 60 °C or 90 °C.
39. The method of any of any of clauses 36 to 38, further comprising overcoating the silica coating with an organic coating, optionally wherein the organic coating comprises a spacer and/or a polymer.
40. The method of clause 39, wherein the overcoating is performed in the presence of a water miscible solvent, optionally wherein the water miscible solvent is Cyrene™.
41. The method of any of clauses 28 to 40, wherein the coating comprises functional groups;
   optionally wherein the functional groups are selected from hydroxyl groups, carboxylic acid groups, aldehyde groups, amine groups, thiol groups, epoxy groups, amide groups, chloromethyl groups, and tosyl-activated groups;
   further optionally wherein the functional groups are selected from a hydroxyl, a carboxylic acid (—COOH), a primary amine, a secondary amine, or epoxy; further optionally wherein the functional groups are selected from a carboxylic acid (—COOH), a primary amine, a secondary amine, or an epoxy.
42. The method of any of clauses 28 to 41, wherein the magnetic clusters comprise nanocrystals of iron oxide comprising an average diameter of from about 5 nm to about 40 nm, optionally wherein the nanocrystals have a diameter of from about 5 nm to about 20 nm, further optionally wherein the nanocrystals have a diameter of from about 10 nm to about 15 nm, still further optionally wherein the nanocrystals have a diameter of about 12 nm.
43. The method of any of clauses 28 to 42, wherein the magnetic clusters are monodisperse.
44. The method of any of clauses 28 to 43, wherein the magnetic clusters have a diameter in the range of from about 100 nm to about 2,500 nm;
   optionally wherein the magnetic clusters have a diameter in the range of from about 200 nm to about 1,000 nm.
45. Magnetic clusters obtainable by the method of any of clauses 28 to 44.
46. Use of magnetic clusters of any of clauses 1 to 27, or of clause 45 in an assay.
47. Use of magnetic clusters of any of clauses 1 to 27, or of clause 45 for isolating, purifying, fractionating, depleting, concentrating and/or analysing cells, viruses, vesicles, organelles, exosomes, small molecules, analytes cations, anions, ions, toxins, pathogens, nucleic acids, proteins or peptides.
48. The use of clause 46 or 47, wherein the magnetic clusters comprise a (surface) capture ligand, optionally wherein the capture ligand is selected from the group consisting of antibodies, antibody fragments, peptides, carbohydrates, haptens, aptamers, and oligonucleotides.
49. The use of clause 46 or 47, wherein the magnetic clusters comprise surface functionalization for capture of a class of molecules, optionally wherein the surface functionalization comprises ionic groups or hydrophobic groups.
50. The use of any of clauses 46 or 48-49, wherein the assay comprises immunoprecipitation, nucleic acid capture, immunoassay, and/or lateral flow assay.
51. A kit, comprising:
   magnetic clusters of any of clauses 1 to 27, or of clause 45.
52. The kit of clause 51, further comprising one or more buffers.
53. The kit of clause 52, wherein the one or more buffers comprise a lysis and/or binding buffer, and optionally a washing buffer and/or an elution buffer
54. The kit of clause 53, wherein the lysis and/or binding buffer may contain one or more of a chaotrope, a precipitating agent such as an alcohol or an ethylene glycol.

Additional embodiments are provided by the following numbered clauses:
1. Magnetic clusters, wherein the magnetic clusters comprise nanocrystals of iron oxide and an aromatic carboxylic acid.
2. The magnetic clusters of clause 1, wherein the aromatic carboxylic acid is a compound of formula (I):

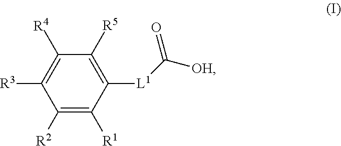

wherein:
L¹ is selected from a bond, —C₁₋₆alkyl-, or —C₂₋₆alkenyl-;
R¹ is selected from —H, —COOH, —(C₁₋₅alkyl)COOH, —(C₂₋₅alkenyl)COOH, or —C₁₋₄alkoxy;
R² is selected from —H, —C₁₋₄alkyl, —C₁₋₄alkoxy;
R³ is selected from —H, -halo, —C₁₋₆alkyl, —C₁₋₆alkoxy, -halo(C₁₋₆)alkyl, —NR⁶R⁷, or substituted or unsubstituted phenyl;
R⁴ is selected from —H, —C₁₋₄alkyl, —C₁₋₄alkoxy;
R⁵ is selected from —H, or —C₁₋₄alkoxy;
R⁶ is selected from —H, or —C₁₋₄alkyl; and
R⁷ is selected from —H, —C₁₋₄alkyl, —C(O)H, —C(O)C₁₋₄alkyl.
3. The magnetic clusters of clause 2, wherein L¹ is selected from a bond, —C₁₋₄alkyl-, or —C₂-4alkenyl-;
   optionally wherein L¹ is selected from a bond or a —C₂₋₄alkenyl-.
4. The magnetic clusters of clause 2 or clause 3, wherein R¹ is selected from —H, —COOH, —(C₁₋₅alkyl)COOH, or —(C₂₋₅alkenyl)COOH;
   optionally wherein R¹ is selected from —H, or —COOH.
5. The magnetic clusters of any of clauses 2 to 4, wherein R² is selected from —H, or —C₁. 4alkoxy;
   optionally wherein R² is selected from —H, or —OCH₃.
6. The magnetic clusters of any of clauses 2 to 5, wherein R³ is selected from —H, -halo, —C₁. 4alkyl, —C₁₋₄alkoxy, -halo(C₁₋₄)alkyl, —NR⁶R⁷, or substituted or unsubstituted phenyl;
   optionally wherein R³ is selected from —C₁₋₄alkoxy, -halo(C₁₋₄)alkyl, —NR⁶R⁷, or substituted or unsubstituted phenyl.
7. The magnetic clusters of any of clauses 2 to 6, wherein R⁶ is —H or —CH₃, and/or wherein R⁷ is —CH₃ or —C(O)CH₃.
8. The magnetic clusters of any of clauses 2 to 7, wherein R⁴ is selected from —H, or —C₁. 4alkoxy;
   optionally wherein R⁴ is selected from —H, or —OCH₃.
9. The magnetic clusters of any of clauses 2 to 8, wherein R⁵ is —H; and/or
   wherein R¹ and R⁵ are both —H; and/or
   wherein R³ is —OH, R² is —C₁₋₄alkoxy, and R³ is —C₁₋₄alkoxy.
10. The magnetic clusters of any preceding clause, wherein the aromatic carboxylic acid is selected from benzoic acid, toluic acid, 4-ethylbenzoic acid, 3,5-dimethylbenzoic acid, 4-t-butylbenzoic acid, p-anisic acid, 3,4,5-trimethoxybenzoic acid (eudesmic acid), trans-cinnamic acid, phthalic acid, homophthalic acid, 4-(trifluormethyl)benzoic acid, 4-iodobenzoic acid, 4-chlorobenzoic acid, 3-phenylpropionic acid, 4-(dimethylamino)benzoic acid, 4-acetamidobenzoic acid, bisphenyl-4-carboxylic acid.
11. The magnetic clusters of any proceeding clause, wherein the magnetic clusters are monodisperse.
12. The magnetic clusters of any preceding clause, wherein the magnetic clusters have a mode diameter in the range of from about 100 nm to about 2,500 nm;
   optionally wherein the magnetic clusters have a mode diameter in the range of from about 200 nm to about 1,000 nm.
13. Monodisperse magnetic clusters comprising nanocrystals of iron oxide, wherein the magnetic clusters have a mode diameter in the range of from about 100 nm to about 2,500 nm and a coefficient of variation (CV) of not more than 5% when measured by disc centrifuge.
14. The monodisperse magnetic clusters of clause 13, wherein the magnetic clusters further comprise an aromatic carboxylic acid, optionally wherein the aromatic carboxylic acid is as defined in any of clauses 2 to 12.
15. The magnetic clusters of any of clauses 1 to 12, or monodisperse magnetic clusters of clause 13 or clause 14, wherein the nanocrystals of iron oxide comprise a mode average diameter of from about 5 nm to about 20 nm, optionally wherein the nanocrystals have a mode average diameter of from about 10 nm to about 15 nm, further optionally wherein the nanocrystals have a mode average diameter of about 12 nm.
16. The magnetic clusters of any of clauses 1 to 12 or 15, or monodisperse magnetic clusters of any of clauses 13 to 16, further comprising a coating.
17. The magnetic clusters or monodisperse magnetic clusters of clause 16, wherein the coating comprises at least one layer of silica; and/or wherein the coating comprises at least one layer of organic coating, optionally wherein the at least one layer of organic coating overcoats at least one layer of silica.
18. The magnetic clusters or monodisperse magnetic clusters of clause 16 or clause 17, wherein the coating comprises functional groups;
   optionally wherein the functional groups are selected from hydroxyl groups, carboxylic acid groups, aldehyde groups, amine groups, thiol groups, epoxy groups, amide groups, chloromethyl groups, and tosyl-activated groups;
   further optionally wherein the functional groups are selected from a hydroxyl, a carboxylic acid (—COOH), a primary amine, a secondary amine, or epoxy; further optionally wherein the functional groups are selected from a carboxylic acid (—COOH), a primary amine, a secondary amine, or an epoxy.
19. A method of making magnetic clusters, comprising:
   forming a solution comprising iron (III) ions, a protic solvent, an aromatic carboxylic acid, and a precipitator; and allowing the solution to react to form a suspension comprising magnetic clusters.
20. The method of clause 19, wherein the protic solvent is an alcohol, optionally a diol or polyol, further optionally wherein the alcohol is ethylene glycol.
21. The method of clause 19 or clause 20, wherein the aromatic carboxylic acid is as defined in any of clauses 2 to 10.
22. The method of any of clauses 19 to 21, wherein the precipitator is an acetate, optionally sodium acetate.
23. The method of any of clauses 19 to 22, further comprising separating the magnetic clusters from the suspension;
   optionally further comprising washing the magnetic clusters.
24. The method of clause 23, further comprising adding a silica coating to the separated magnetic clusters, comprising:
   forming a suspension comprising the separated magnetic clusters and silicates or orthosilicates; and
reacting the silicates or orthosilicates to form a silica coating.
25. The method of clause 24, further comprising overcoating the silica coating with an organic coating, optionally wherein the organic coating comprises a spacer and/or a polymer.
26. The method of any of clauses 19 to 25, wherein the coating comprises functional groups;
   optionally wherein the functional groups are selected from hydroxyl groups, carboxylic acid groups, aldehyde groups, amine groups, thiol groups, epoxy groups, amide groups, chloromethyl groups, and tosyl-activated groups;
   further optionally wherein the functional groups are selected from a hydroxyl, a carboxylic acid (—COOH), a primary amine, a secondary amine, or epoxy; further optionally wherein the functional groups are selected from a carboxylic acid (—COOH), a primary amine, a secondary amine, or an epoxy.
27. The method of any of clauses 19 to 26, wherein the magnetic clusters are monodisperse; and/or
   wherein the magnetic clusters have a diameter in the range of from about 100 nm to about 2,500 nm;
      optionally wherein the magnetic clusters have a diameter in the range of from about 200 nm to about 1,000 nm.
28. Use of magnetic clusters of any of clauses 1 to 18 in an assay.
29. The use of clause 28, wherein the magnetic clusters comprise a (surface) capture ligand, optionally wherein the capture ligand is selected from the group consisting of antibodies, antibody fragments, peptides, carbohydrates, haptens, aptamers, and oligonucleotides; and/or
   wherein the magnetic clusters comprise surface functionalization for capture of a class of molecules, optionally wherein the surface functionalization comprises ionic groups or hydrophobic groups; and/or
   wherein the assay comprises immunoprecipitation, nucleic acid capture, immunoassay, and/or lateral flow assay.
30. A kit, comprising:
   magnetic clusters of any of clauses 1 to 18.
31. The kit of clause 30, further comprising one or more buffers;
   optionally wherein the one or more buffers comprise a lysis and/or binding buffer, and
optionally a washing buffer and/or an elution buffer.

### Analytical Methods

### Measurement of Particle Size and CV by CPS Disc Centrifuge

The size distribution of samples can be measured using disc centrifugation, e.g. CPS Disc Centrifugation™ on Disc Centrifuge Model DC20000, using protocols provided by the instrument manufacturer. Accurate results require calibration with a standard of similar density to the sample being analysed, e.g. a set of compact polystyrene particle standards could be used for particles that predominantly comprise polystyrene. Where the samples being measured have a density that is not known, or where a similar density standard is not available, the measurement obtained by CPS disc centrifugation will be reproducible and thus useful for determining CV but will not provide the actual diameter.

An outline of the CPS Disc Centrifugation™ method that was used in the examples provided herein is as follows. The skilled person would be able to readily adapt these methods as appropriate, for example by selecting a suitable gradient, disc speed and standard particles, based on the size of the particles to be analysed.

Disc centrifuge analysis was performed on a CPS DC20000 from CPS instruments with a disc speed of 5000 rpm and a gradient of 3-7 wt % sucrose in trisodium citrate dihydrate (Na₃Cit·2H₂O) solution (2 mM aqueous). The gradient was made using an Auto Gradient pump from CPS instruments and the volume of the injected gradient was 16-17 mL. The samples were diluted to approximately 0.01 wt % (or as low as 0.0025 wt % to reduce secondary peak artefacts) in MilliQ-H₂O prior to injection. The method used for analysis had the following settings: Max. diameter 3.0 μm, min. diameter 0.2 μm, particles density 3.5 g/mL, particle refractive index 2.344, particle absorption 0.13, particle non-sphericity 1, calibration standard diameter 1.099 μm, calibration standard density 1.6, standard half-with 0.2 μm, liquid density 1.016 g/mL, liquid refractive index 1.343.

We have observed that insufficient dilution of the sample may result in secondary peak artefacts appearing as apparent larger particle sizes, due to two or more (e.g. 2, 3, 4, or more) of the particles clumping during the disc centrifuge analysis. Such artefacts may artificially increase the CV and/or particle diameter determined from the disc centrifuge analysis. Accordingly, where secondary peak artefacts are suspected, disc centrifuge analysis is preferably performed at a relatively high sample dilution, e.g. the sample may be diluted to as low as approximately 0.0025 wt %.

Suitable particles for use as a standard for disc centrifuge analysis of particles of the invention are crosslinked polystyrene particles. For instance, in some examples provided herein, the standard particles used were magnetisable porous 1 μm polystyrene particles cross-linked with 50% divinylbenzene, coated with a crosslinked hydrophilic polyether. The standard particles had a density of about 1.6 g/cm³ in water.

The size reported is based on the mode absorption peak diameter. The CV is determined by taking the upper and lower diameters of the mode peak at its half height (also known as the half height width), dividing it by the mode peak diameter and multiplying by 100 to provide a CV (%).

Use of the mode peak at its half height for determination of CV may be advantageous, as it reduces the risk that secondary or tertiary peak artefacts affect the determination of CV. Such artefacts may be due to agglomeration of a minority of the particles during the disc centrifuge analysis.

### Measurement of Aggregation and Agglomeration Level, Size and Morphology of Magnetic Clusters by Optical Microscopy

Qualitative characterization of the magnetic clusters may be performed by optical microscopy. Images obtained with optical microscopy may be used to investigate the particle aggregation and agglomeration level (AL), and give an indication of the size. The resolution of optical microscopy is, however, not high enough to provide good images of particles with a size/diameter below 1 μm.

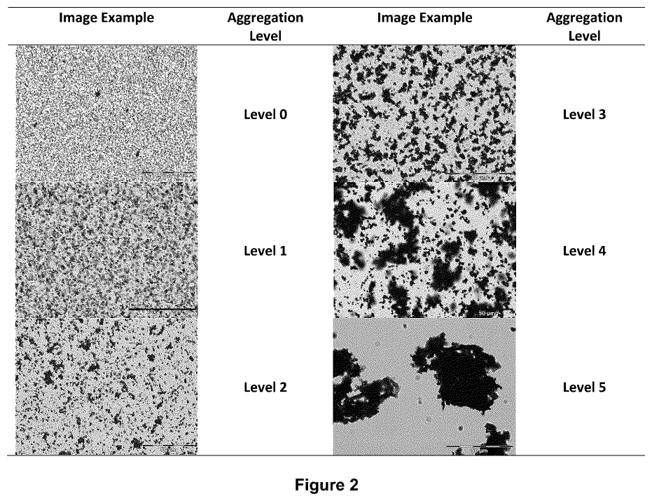

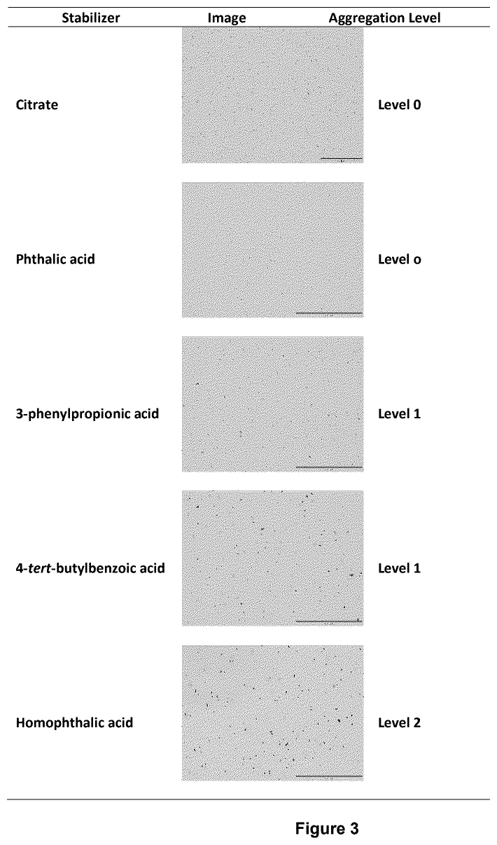

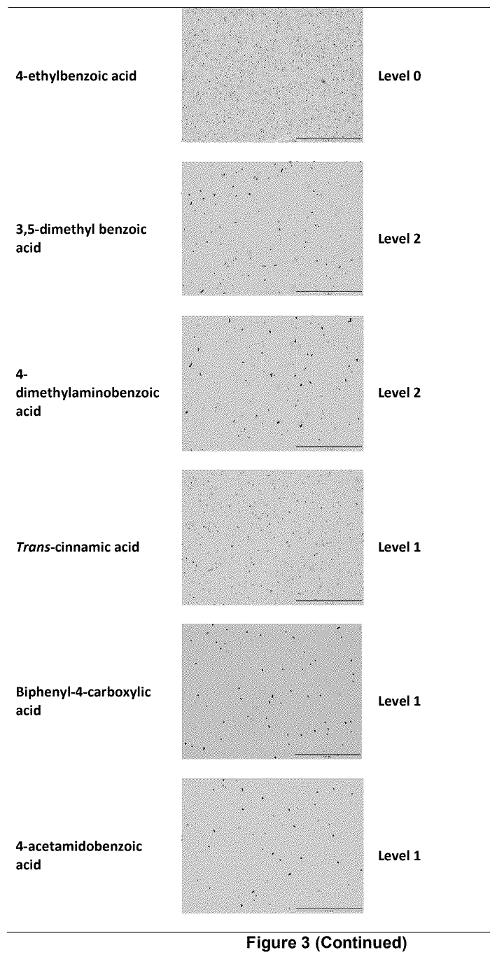

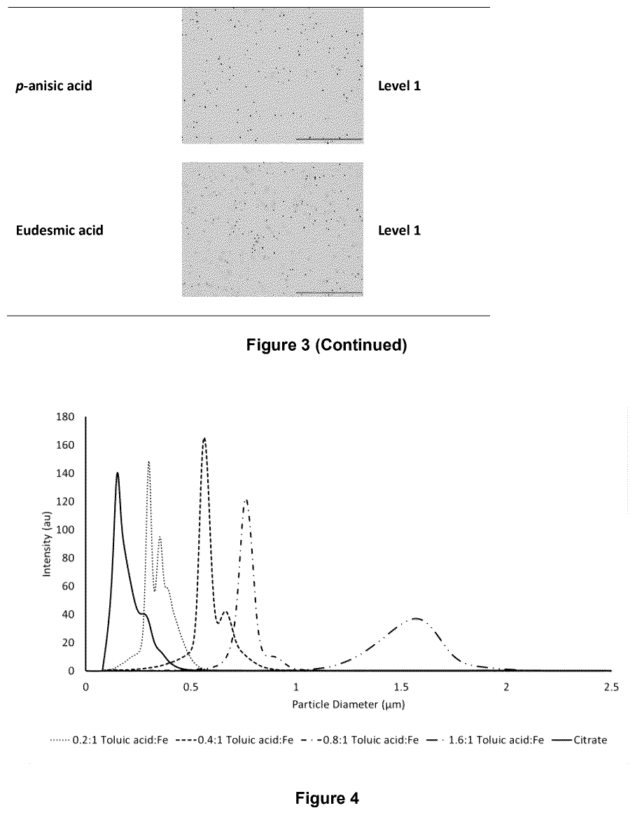

Batches of magnetic clusters (or other particles) imaged at a 1.6 times 10×, 20× and 40× (100×) magnification as a H₂O-suspension either undiluted or further diluted in H₂O. The aggregation and agglomeration level of the batches were determined visually and characterized using the 5 level scale illustrated in FIG. 2, while exemplary results for the batches is provided in FIG. 3. The scale bar in the bottom right of each of the individual figures represents 50 μm.

### Measurement of Cluster Size by Scanning Electron Microscopy (SEM)

Magnetic clusters (including those with a size/diameter below 1 μm) may be imaged by SEM to obtain high resolution images for accurate determination of the diameter of the particles.

A suitable method of SEM analysis is as follows. Magnetic clusters may be provided as an aqueous suspension, for example with a particle dry content of about 5%, or as vacuum dried particles. Before analysis, a drop of the aqueous suspension was deposited on carbon tape and left to dry under a warm 100 W lamp. Preferentially the particles (dried under vacuum) are trapped in epoxy resin, which is then cured at 60 °C for 1-2 days. The SEM images may be taken using any suitable SEM instrument at an appropriate level of magnification. For analysis of the particles of the examples, the SEM instrument was a Hitachi S-4800 FEG, with a magnification between 5 000 and 100 000× at a V_{acc} of 2 kV and a working distance of 2.8 mm.

The average size of particles in an image may be quantified in the following manner. The images were analyzed using the open platform image processing program “Fiji Is Just—Image J” (Schindelin, J., et al. (2012). Fiji: an open-source platform for biological-image analysis. Nature Methods, 9(7), 676-682). Using the scale bar for calibration, at least 200 particles were manually selected for each batch before the number average and surface average size was calculated.

### Measurement of Magnetic Properties

Magnetic properties of magnetic nanoclusters, such as remnant mass magnetization, saturation mass magnetization and initial magnetic mass susceptibility may be measured in accordance with procedures explained in the standard Nanotechnologies—magnetic nanomaterials Part 2, ISO/TS 19807-2:2021(E) at 5.2.6 and 5.2.7.

### Remnant Mass Magnetization

The remanent mass magnetization of a magnetic cluster is directly proportional to the mechanical force acting on the cluster when it is in a magnetic gradient field at zero absolute magnetic field. Thus, remanent mass magnetization can affect the speed and effectiveness of the extraction process in many biological assays, as well as the possible agglomeration of beads. When solid magnetizable surfaces are present in the reaction environment, the remanent mass magnetization can also lead to unwanted accumulation of the clusters at these surfaces in the absence of an external magnetic field.

For the measurement of the remanent mass magnetization, the magnetic clusters should be washed and dried in an oven. Their mass is determined by weighing. The magnetic moment of the dried clusters sample is measured using a suitable device, such as a superconducting quantum interference device (SQUID) or a vibrating-sample magnetometer (VSM). During the measurement, the dried magnetic clusters sample is first exposed to a high magnetic field in the range of the saturation magnetization field. Then, the magnetic field is monotonically brought to zero and the remaining magnetic moment of the sample is measured. The remanent mass magnetization is calculated as the ratio of the magnetic moment at zero field and the mass of the magnetic beads and reported using the unit A·m²/kg.

### Saturation Mass Magnetization

The saturation mass magnetization of magnetic clusters has an influence on the speed and effectiveness of the extraction process during assays and on the possible agglomeration of beads during the extraction process. It is an important characteristic of the magnetic behaviour of the clusters and can be used to monitor the quality of the clusters.

For the measurement of the saturation mass magnetization, the magnetic clusters should be washed and dried in an oven. Their mass is determined by weighing. The magnetic moment of the dried clusters sample is measured using a suitable device, such as a SQUID or a VSM. During the measurement, the dried magnetic clusters sample is susceptible to an increasing magnetic field until the value of the magnetic moment is no longer changing with field increase. At this field strength, the magnetic moment of the sample is measured. The saturation mass magnetization is calculated as the ratio of the measured magnetic moment and the mass of the magnetic beads and reported using the unit A-m²/kg.

### Initial Magnetic Mass Susceptibility

The initial magnetic mass susceptibility is an important characteristic of magnetic clusters for use in assays, such as biological assays that include at least one extraction step, because it can have a significant influence on the extraction performance. In the absence of an external magnetic field, the net magnetization of the magnetic clusters is small due to random orientation of the magnetic moments of the nanoparticles inside the clusters. However, when an external magnetic field is switched on, the magnetic moments of the nanoparticles inside the clusters will acquire a preferential orientation and thus the magnetic clusters will develop a net magnetization. The ratio between the change in magnetization and the corresponding change in magnetic field is called magnetic susceptibility. The susceptibility that is measured at a very small absolute magnetic field is called initial magnetic susceptibility.

Before the measurement, the magnetic clusters should be washed and dried in an oven. Their mass is determined by weighing. For the measurement of the initial magnetic susceptibility, the dried magnetic clusters sample should be demagnetized by a sufficiently small absolute magnetic field with vanishing amplitude over time. Then, the magnetic moment of the dried magnetic clusters sample is measured using VSM or a SQUID at a small absolute magnetic field amplitude, where the relation between the magnetic moment of the sample and the magnetic field is still sufficiently linear. The initial magnetic mass susceptibility of a magnetic clusters sample is calculated by dividing the magnetic moment of the sample by the product of the applied magnetic field and the mass of the dried clusters. The result of the measurement is expressed in the unit m³/kg.

### Detection of Aromatic Carboxylic Acid in Magnetic Clusters

The presence and/or identity of the aromatic carboxylic acid use as stabilizers in the magnetic clusters of the invention may be confirmed using a suitable mass spectrometry method, such as analysis of the magnetic clusters using electrospray ionization mass spectrometry. An exemplary method is provided below.

A sample of magnetic clusters were washed and decanted on magnet with 96% Ethanol (3 times), followed by water (3 times). After removal of the solvent, the magnetic clusters were dissolved in a solution of Hydrochloric acid. The acid was subsequently diluted with water, and the aqueous phase was extracted three times with chloroform. The organic phases were combined and evaporated in vacuo to provide a sample. The sample was dissolved in a suitable solvent, with the resulting solution then introduced into the ESI-MS instrument. The presence and/or identity of the aromatic carboxylic acid(s) may then be confirmed by a skilled operator using, e.g. high resolution analysis or tandem MS/MS analysis.

Where the sample of magnetic clusters are coated, the coating can be removed or disrupted using standard methods prior to the analysis described in the above paragraph. For example, for silica coated particles, the silica coating may be removed by reaction with a basic solution before performing the analysis.

### Measurement of Specific Surface Area

Specific surface area is a characteristic of finely divided samples, such as magnetic clusters. Determination of specific surface area rely on measuring the amount of gas adsorbed at different partial pressures. Adsorption can happen in two ways: physical and chemical adsorption. Physical adsorption (or physisorption) is weaker than chemical adsorption (chemisorption), and is only observed at low temperatures. This tends to mean that physisorption tends to be reversible when the partial pressure is decreased. The adsorption and desorption of physiosorbed gas molecules is what is measured during specific surface area measurements.

Specific surface area may be measured in accordance with a Brunauer-Emmett-Teller (BET) method, for example as set out in a relevant standard (e.g. Determination of the specific surface area of solids by gas adsorption—BET method, Second edition, BS ISO 9277:2010). An exemplary procedure as used herein is provided below.

Preparation of samples for measurements is done using Micromeritics VacPrep 061. Measurements are performed using a Micromeritics TriStar 300 Surface Area and Porosimetry Analyzer with Argon as the analysis molecule.

First the sample tube and filler rod are weighed. Then an amount of pre-weighed sample between 0.2500 and 0.5000 g is weighed and added (ensuring the sample goes all the way down to the bottom of the glass tube). The filler rod is placed in the sample tube and then the sample tube with filler rod and sample is weighed. The weighed samples are then attached to a vacuum preparation station evacuated and then treated at 160 °C for 16-18 hours. When the vacuum preparation is finished the sample tubes are reweighed before being attached to the analysis port and the Dewar flask filled with liquid nitrogen. The program is then run with adsorption and desorption points chosen based on the samples that are being measured. For non-porous and mesoporous materials it is important for BET measurement to measure where the linear region is found between (p/p0)=0.05 and 0.35.

### EXAMPLES

### Synthesis of Magnetic Clusters

### Example 1

Ethylene glycol (150 mL) and Iron(III)chloride hexahydrate (8.11 g, 30 mmol) were charged to a conical flask and stirred for 15 minutes. Then sodium citrate (1.28 g, 6 mmol) was then added, the flask sealed, flushed with argon and stirred for 15 mins. The mixture was then sonicated for 15 minutes, after which, Sodium acetate (8.61 g, 105 mmol) was added and the solution was stirred for 15 minutes (under argon), subsequently sonicated for 30 minutes, stirred for 15 minutes, and sonicated for a final 30 minutes. The solution was thereafter charged to a Teflon liner, blanketed with argon, sealed and placed in a steel-lined autoclave. The autoclave was heated to 210 °C over the course of 60 minutes, maintained at 210 °C for 10 hours, and then allowed to cool to room temperature. The reaction was filtered with a wire mesh to remove any large particulates and transferred to a bottle using ethanol (96%). The suspension was washed and decanted on magnet with 96% Ethanol (3 times), water (3 times), once with 1 M sodium citrate solution (including 30 min shaking and 15 min sonication), and finally with water (3 times).

### Example 2

Ethylene glycol (150 mL) and Iron(III)chloride hexahydrate (8.11 g, 30 mmol) were charged to a conical flask and stirred for 15 minutes. Then p-toluic acid (3.27 g, 24 mmol) was then added, the flask sealed, flushed with argon and stirred for 15 mins. The mixture was then sonicated for 15 minutes, after which, Sodium acetate (8.61 g, 105 mmol) was added and the solution was stirred for 15 minutes (under argon), subsequently sonicated for 30 minutes, stirred for 15 minutes, and sonicated for a final 30 minutes. The solution was thereafter charged to a Teflon liner, blanketed with argon, sealed and placed in a steel-lined autoclave. The autoclave was heated to 210 °C over the course of 60 minutes, maintained at 210 °C for 10 hours, and then allowed to cool to room temperature. The reaction was filtered with a wire mesh to remove any large particulates and transferred to a bottle using ethanol (96%). The suspension was washed and decanted on magnet with 96% Ethanol (3 times), water (3 times), once with 1 M sodium citrate solution (including 30 min shaking and 15 min sonication), and finally with water (3 times).

The presence of toluic acid in the resulting magnetic clusters was confirmed using a high resolution analysis on an ESI-MS instrument in accordance with the method of detection of aromatic carboxylic acid in magnetic clusters provided herein.

This synthesis method was repeated, but with the mol ratio of toluic acid to Iron(III)chloride adjusted to levels of 0.2, 0.4 and 1.6. The magnetic clusters obtained in Example 2 (toluic acid stabilizer) and Example 1 (citric acid stabilizer) were analysed by disc centrifuge, with the results indicated in FIG. 4. This demonstrated that increasing the concentration of toluic acid relative to Iron(III)chloride increased size of particles. In addition, use of toluic acid as a stabilizer provided magnetic nanoclusters with a smaller CV than when citric acid was used as a stabilizer.

TABLE 1
Synthesis with toluic acid as a stabilizer

| | Mass or | | | Mol | | |
|---|---|---|---|---|---|---|
| Reagents | Volume | | mmol | comparison | g/mol | mol/L |
| Ethylene Glycol | 150 | mL | 2683 | 89.4 | 62.07 | 17.88 |
| Iron(III)chloride | 8.11 | g | 30.00 | 1.00 | 270.3 | 0.200 |
| hexahydrate | | | | | | |
| Toluic acid | 3.27 | g | 24.00 | 0.80 | 128.2 | 0.160 |
| Sodium Acetate | 8.61 | g | 5.63 | 3.5 | 3.50 | 0.70 |

### Examples 3-15

In Examples 3 to 15, the procedure of Example 2 was followed, but with a different organic aromatic acid as the stabilizer.

In a typical procedure, ethylene glycol (150 mL) and Iron(III)chloride hexahydrate (8.11 g, 30 mmol) were charged to a conical flask and stirred for 15 minutes. Then the stabilizer (24 mmol, masses listed in Table 2) was then added, the flask sealed, flushed with argon and stirred for 15 mins. The mixture was then sonicated for 15 minutes, after which, Sodium acetate (8.61 g, 105 mmol) was added and the solution was stirred for 15 minutes (under argon), subsequently sonicated for 30 minutes, stirred for 15 minutes, and sonicated for a final 30 minutes. The solution was thereafter charged to a Teflon liner, blanketed with argon, sealed and placed in a steel-lined autoclave. The autoclave was heated to 210 °C over the course of 60 minutes, maintained at 210 °C for 10 hours, and then allowed to cool to room temperature. The reaction was filtered with a wire mesh to remove any large particulates and transferred to a bottle using ethanol (96%). The suspension was washed and decanted on magnet with 96% Ethanol (3 times), water (3 times), once with 1 M sodium citrate solution (including 30 min shaking and 15 min sonication), and finally with water (3 times)

TABLE 2
Stabilizers used in Examples 2-15

| Example | Name of Stabilizer | Mass (g) |
|---|---|---|
| 3 | Eudesmic acid | 5.09 |
| 4 | 4-acetamidonezoic acid | 4.30 |
| 5 | Bisphenyl-4-carboxylic acid | 4.76 |
| 6 | Trans-cinnamic acid | 3.56 |
| 7 | 4-(dimethylamino)benzoic acid | 3.96 |
| 8 | 3,5-dimethylbenzoic acid | 3.60 |
| 9 | 4-ethylbenzoic acid | 3.60 |
| 10 | 4-tertbutylbenzoic acid | 4.28 |
| 11 | Benzoic acid | 2.93 |
| 12 | p-anisic acid | 3.65 |
| 13 | phthalic acid | 3.99 |
| 14 | homophthalic acid | 4.32 |
| 15 | 3-phenylpropionic acid | 3.60 |

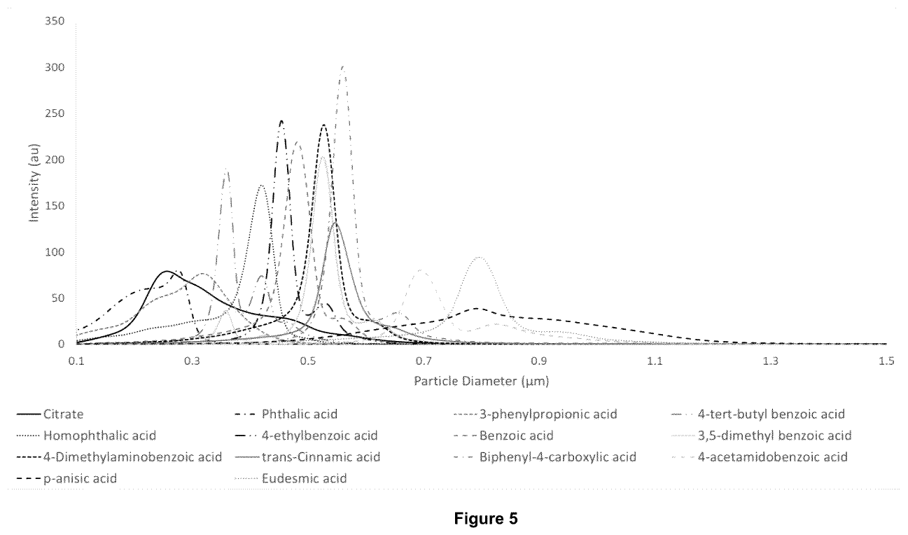

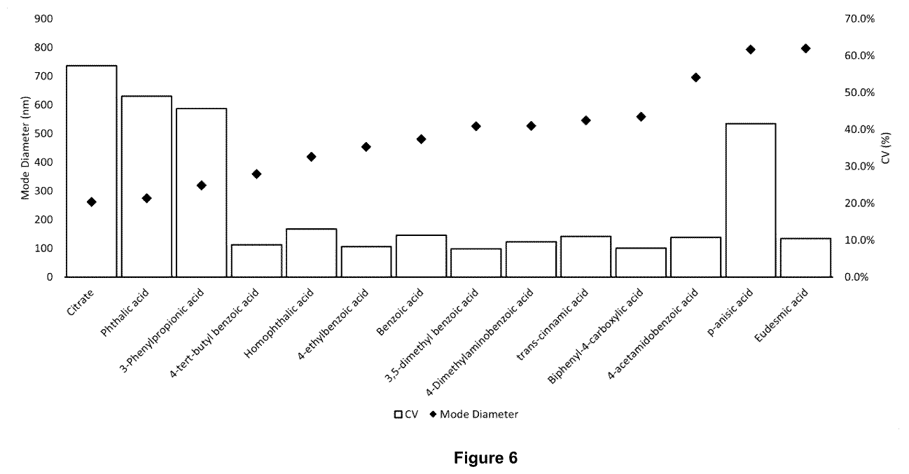

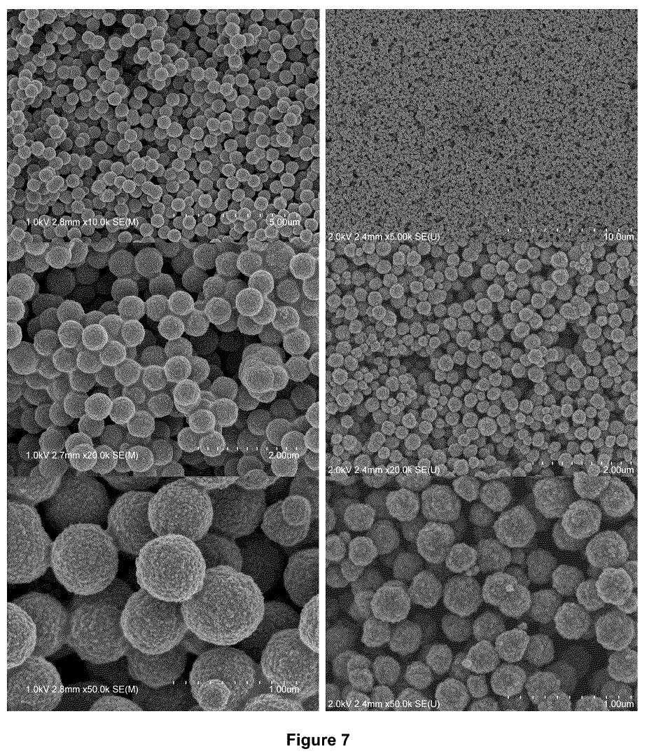

The magnetic clusters obtained were analysed by disc centrifuge, with the results illustrating the mode peak provided in FIG. 5. FIG. 6 provides an indication of the mode diameter of the magnetic clusters (diamond markers) and CV (bars) determined from the disc centrifuge results. The results for the citrate reference example are also included in FIGS. 5 and 6. SEM images (FIG. 7) were also obtained for the magnetic clusters obtained with bisphenyl-4-carboxylic acid (Example 5) and the citrate reference example.

### Examples 16-18

In Examples 16 to 18, the procedure of Example 2 was followed, but with using the following organic aromatic acid as the stabilizer: 4-(Trifluoromethyl)benzoic acid (Example 16), 4-iodobenzoic acid (Example 17), or 4-chlorobenzoic acid (Example 18).

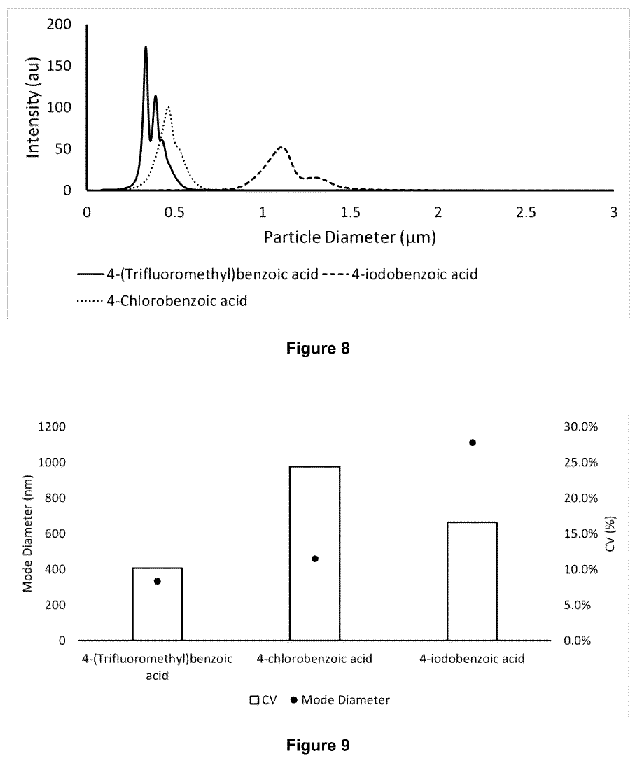

The magnetic clusters obtained were analysed by disc centrifuge, with the results illustrating the mode peak provided in FIG. 8. FIG. 9 provides an indication of the mode diameter of the magnetic clusters (circular markers) and CV (bars) determined from the disc centrifuge results.

### Coating Magnetic Clusters

### Example 19: Silica Coating of Magnetic Clusters

Magnetic clusters in water (2.0 g Dry Content) were washed with EtOH (96%, 3×100 mL) using magnetic separation. The clusters, EtOH (99.8%, 170 mL) and NH₃ (2 mL, 25% in H2O) were added to a round bottom flask before being ultra-sonicated for 15 min. EtOH (99.8%, 30 mL) and TEOS (1.40 mL) were mixed in a separate flask and quickly added to the above suspension. The flask was stirred at room temperature for 18 h. The final product was washed with EtOH (96%, 3×100 mL) and H₂O (5×100 mL) and stored as a H₂O-suspension.

### Example 20: Epoxy Coating on Silica Coated Clusters

Silica coated magnetic clusters in water (2.0 g dry content) were washed with Cyrene™ (3×100 ml) using magnetic separation. The particles and Cyrene™ (16.73 g) were ultrasonicated for 15 minutes. (3-Glycidyloxypropyl)trimethoxy silane (21.27 g) was added, the mixture was heated to 130 °C and reacted for 18 h. After cooling, the epoxy functionalized particles were washed with H₂O (2×100 mL) and Cyrene™ (5×100 mL). The particles were left suspended in Cyrene™ (65 mL).

### Example 21: Coupling of Protein G to Silica-Epoxy Coated Clusters

A suspension of Silica-Epoxy coated clusters in Cyrene™ (10 mg dry weight) were transferred to a 2 mL tube and placed on a magnetic separation device. The supernatant was removed, and the clusters were washed twice in water (1 mL) and once in 0.1 M Phosphate buffer pH 7.4 (Cl) (1 mL) using resuspension by vortexing and sonication for five minutes in a water bath, followed by magnetic separation for each wash. The particles were resuspended in C1 (195 μl). Protein A/G (Thermo Fisher Scientific #77677) in C1 (10 mg/mL, 55 μl) was added to the clusters and the mixture was incubated on a roller for 5 minutes. 3 M Ammonium Sulfate (C2) (250 μl) was added, and the particles incubated on a roller at 37 °C overnight.

The clusters were washed three times with 50 mM Tris, 140 mM NaCl, 0.1% Tween-20 (TBST) (1 mL) as described above and incubated overnight at 37 °C on a roller. The clusters were again washed once in TBST (1 mL) and then re-suspended in TBST to 20 mg/mL and stored at 2-8 °C until use.

### Example 22: Immunoprecipitation (IP) Using Clusters

The Protein G functionalized clusters from Example 21 can be used for IP following the protocol as described for Dynabeads™ Protein G Immunoprecipitation Kit (Thermo Fisher Scientific #10007D) by replacing the beads provided in the kit with the clusters of Example 21.

The process may also be automated using suitable equipment, such as a KingFisher Flex instrument (Thermo Fisher Scientific #5400640) with the script provided for the above-mentioned kit. A brief description of an exemplary manual process is given below.

Sample preparation: Jurkat cells (10×10 6) were lysed by adding RIPA buffer (1000 μl). The sample was incubated for 15 minutes on ice then centrifuged at 14000 g, 4 °C for 15 minutes. The supernatant was carefully transferred to a new tube.

Cluster preparation: Anti-CD81 antibody (Thermo Fisher Scientific #10630D) (0.5 mg/mL, 5 μl) was added to particles from Example 17 (25 μl, 0.5 mg) in PBS with 0.05% Tween-20. Binding of the antibody was achieved by incubation on a roller for 30 minutes, then the supernatant was removed and the particles were washed and resuspended in PBS with 0.05% Tween-20 (200 μl).

Immunoprecipitation: The clusters with antibody bound (200 μl) were placed on a magnet and the supernatant removed. The sample (cell lysate) (200 μl) was added, the particles re-suspended by pipetting and then incubated on a roller for 30 minutes at room temp. The particles were then washed three times using magnetic separation and resuspension by gentle pipetting using PBS with 0.05% Tween-20 (200 μl).

Elution: The supernatant was removed using magnetic separation and elution buffer (LDS sample buffer, Thermo Fisher Scientific #B0007) (25 μl) was added. The particles were re-suspended by pipetting and then incubated at 70 °C for 10 minutes. The particles were separated using a magnet and the eluate transferred to a new tube.

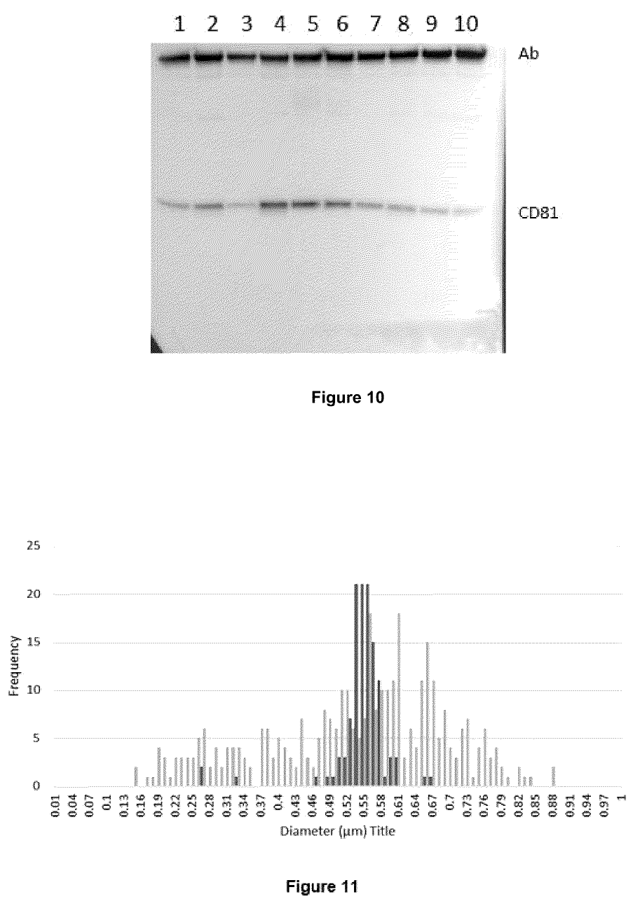

Analysis of the eluate: The eluate was analyzed using precast polyacrylamide gels with Coomassie staining. FIG. 10 provides an image of such a gel at the end of the analysis and Table 3 provides information on the sample loaded into each column of the gel. The column “Bead batch” refers to slight variations in the preparation of the coating and antibody coupling. There is a clean band for the target CD81 antigen in all cases. In addition, the antibody bound by affinity to Protein A/G will be eluted due to the denaturing conditions used.

TABLE 3
Samples loaded to the gel

| sample | Bead batch | Description | Gel 2 wash |
|---|---|---|---|
| 1 | MA236 | Standard buffers + CTAC | TBST + BSA |
| 2 | MA236 | Standard buffers | TBST + BSA |
| 3 | MA406 | Standard buffers + CTAC | TBST + BSA |
| 4 | MA406 | Standard buffers | TBST + BSA |
| 5 | MA407 | Standard buffers + CTAC | TBST + BSA |
| 6 | MA407 | Standard buffers | TBST + BSA |
| 7 | MA371 | Standard buffers + CTAC | TBST + BSA |
| 8 | MA371 | Standard buffers | TBST + BSA |
| 9 | MA372 | Standard buffers + CTAC | TBST + BSA |
| 10 | MA372 | Standard buffers | TBST + BSA |

### Further Synthesis of Magnetic Clusters

### Examples 23-42

In Examples 23-42, the procedure of Example 2 was followed, but with a different organic aromatic acid as the stabilizer.

In a typical procedure, ethylene glycol (150 mL) and Iron(III)chloride hexahydrate (8.11 g, 30 mmol) were charged to a conical flask and stirred for 15 minutes. Then the stabilizer (24 mmol, masses listed in Table 2) was then added, the flask sealed, flushed with argon and stirred for 15 mins. The mixture was then sonicated for 15 minutes, after which, Sodium acetate (8.61 g, 105 mmol) was added and the solution was stirred for 15 minutes (under argon), subsequently sonicated for 30 minutes, stirred for 15 minutes, and sonicated for a final 30 minutes. The solution was thereafter charged to a Teflon liner, blanketed with argon, sealed and placed in a steel-lined autoclave. The autoclave was heated to 210 °C over the course of 60 minutes, maintained at 210 °C for 10 hours, and then allowed to cool to room temperature. The reaction was filtered with a wire mesh to remove any large particulates and transferred to a bottle using ethanol (96%). The suspension was washed and decanted on magnet with 96% Ethanol (3 times), water (3 times), once with 1 M sodium citrate solution (including 30 min shaking and 15 min sonication), and finally with water (3 times).

The magnetic clusters obtained were analysed by disc centrifuge according to the analytical methods provided herein, with samples diluted prior to injection to as low as 0.0025 wt %, to reduce secondary peak artefacts. The mode diameters and CVs were indicated in Table 4.

TABLE 4
Stabilizers used in Examples 23-42

| | | | Mode | |
|---|---|---|---|---|
| | | Mass | Diam. | CV |
| Example | Name of Stabilizer | (g) | (nm) | (%) |
| 23 | 4-dimethylaminobenzoic | 3.96 | 527.0 | 9.5 |
| | acid | | | |
| 24 | 4-nitrobenzoic acid | 4.01 | 389.8 | 9.9 |
| 25 | Disodium terephthalate | 5.04 | 265.6 | 10.4 |
| 26 | 4-bromobenzoic acid | 4.82 | 901.6 | 7.8 |
| 27 | 3,5-dimethyl-p-anisic acid | 4.32 | 500.6 | 8.5 |
| 28 | 4-cycloxybenzoic acid | 4.90 | 459.2 | 8.4 |
| 29 | 4-n-pentylbenzoic acid | 4.61 | 331.7 | 9.9 |
| 30 | 4-n-heptylbenzoic acid | 5.29 | 287.7 | 14.4 |
| 31 | 4-n-hexylbenzoic acid | 4.95 | 278.3 | 18.8 |
| 32 | 4-n-butylbenzoic acid | 4.28 | 250.6 | 17.9 |
| 33 | 2-Naptholic acid | 4.13 | 620.0 | 9.2 |
| 34 | 4-isopropylbenzoic acid | 3.94 | 419.6 | 8.2 |
| 35 | Methyl hydrogen | 4.32 | 404.6 | 12.4 |
| | terephthalate | | | |
| 36 | 3-dimethylaminobenzoic | 3.96 | 608.7 | 12.3 |
| | acid | | | |
| 37 | 4-methyl-3,5- | 5.43 | 210.7 | 26.8 |
| | dinitrobenzoic acid | | | |
| 38 | 3-bromo benzoic acid | 4.82 | 722.2 | 9.3 |
| 39 | 4-aminobenzoic acid | 3.29 | 470.5 | 9.3 |
| 40 | 4-(decycloxy)benzoic acid | 2.93 | 541 | 23.2 |
| 41 | 3-bromobenzoic acid | 4.82 | 620.2 | 9.2 |
| 42 | 1-naptholic acid | 4.13 | 525 | 16.9 |

### Example 43

In Example 43, the procedure of Example 2 was followed, but with a lower concentration of organic aromatic acid as the stabilizer. The mode diameter and CV are indicated in Table 5.

TABLE 5
Stabilizers used in Example 43

| | | | Mode | |
|---|---|---|---|---|
| | | Mass | Diameter | CV |
| Example | Name of Stabilizer | (g) | (nm) | (%) |
| 35 | Terepthalic acid | 1.99 | 884.8 | 11.4 |

### Further Analysis of Magnetic Clusters

### Example 44

The effect of the concentration of the stabilizer on the size of the clusters was further investigated, for different concentrations of stabilizers for the stabilizers used in Example 5 (biphenyl-4-carboxylic acid), Example 2 (toluic acid), and Example 35 (4-tert-butylbenzoic acid). The clusters obtained were analysed by disc centrifuge, with the results indicated in Table 6. This demonstrated that, for each particular aromatic carboxylic acid, increasing the concentration of the relevant aromatic carboxylic acid relative to Iron(III)chloride tended to increase the size of particles. The mode diameter was, however, also dependent on the identity of the aromatic carboxylic acid. The mode diameter of the magnetic clusters obtained may accordingly be adjusted by selecting an appropriate aromatic carboxylic acid and adjusting the concentration of the aromatic carboxylic acid to iron(III) ions.

TABLE 6
Effect of change in stabilizer concentration on particle size

| | | Stabilizer | Mode | |
|---|---|---|---|---|
| Fe | | mol/mol | Diameter | CV |
| mol/L | Stabilizer | Fe | (nm) | (%) |
| 0.2 | Biphenyl-4- | 0.1 | 329.0 | 12.5% |
| | carboxylic acid | | | |
| 0.2 | Biphenyl-4- | 0.2 | 335.1 | 11.1% |
| | carboxylic acid | | | |
| 0.2 | Biphenyl-4- | 0.4 | 363.6 | 8.7% |
| | carboxylic acid | | | |
| 0.2 | Biphenyl-4 | 0.8 | 499.5 | 7.0% |
| | carboxylic acid | | | |
| 0.2 | Biphenyl-4 | 1.6 | 531.2 | 6.2% |
| | carboxylic acid | | | |
| 0.2 | Toluic acid | 0.1 | 300.9 | 14.5% |
| 0.2 | Toluic acid | 0.2 | 285.5 | 12.2% |
| 0.2 | Toluic acid | 0.4 | 374.0 | 8.9% |
| 0.2 | Toluic acid | 0.8 | 491.8 | 8.9% |
| 0.2 | Toluic acid | 1.6 | 642.0 | 9.1% |
| 0.2 | 4-tert- | 0.2 | 256.4 | 11.3% |
| | butylbenzoic | | | |
| | acid | | | |
| 0.2 | 4-tert- | 0.4 | 314.6 | 9.2% |
| | butylbenzoic | | | |
| | acid | | | |
| 0.2 | 4-tert- | 0.8 | 359.0 | 8.7% |
| | butylbenzoic | | | |
| | acid | | | |
| 0.2 | 4-tert- | 1.6 | 385.8 | 11.0% |
| | butylbenzoic | | | |
| | acid | | | |

### Example 45

The SEM images of FIG. 7 were analysed to determine the sizes of the clusters obtained with bisphenyl-4-carboxylic acid (Example 5) and the citrate reference example, using the procedure outlined in the Analytical Methods. The results are provided in FIG. 11. The histogram for bisphenyl-4-carboxylic acid is indicated by the dark grey columns, while the monosodium citrate histogram is indicated in light grey. This further confirms that the clusters obtained using bisphenyl-4-carboxylic acid are significantly more monodisperse than the clusters obtained using citrate.

### Example 46

The specific surface area of the magnetic clusters of Examples 2, 10 and 5 were measured in accordance with the exemplary analytical procedure provided herein. The results obtained are indicated in Table 7.

TABLE 7
Specific surface area measurements

| Sample | Name of Stabilizer | Surface area (m²/g) |
|---|---|---|
| Example 2 | Toluic acid | 20.9 |
| Example 5 | Bisphenyl-4-carboxylic acid | 52.1 |
| Example 10 | 4-tertbutylbenzoic acid | 33.3 |

### Example 47

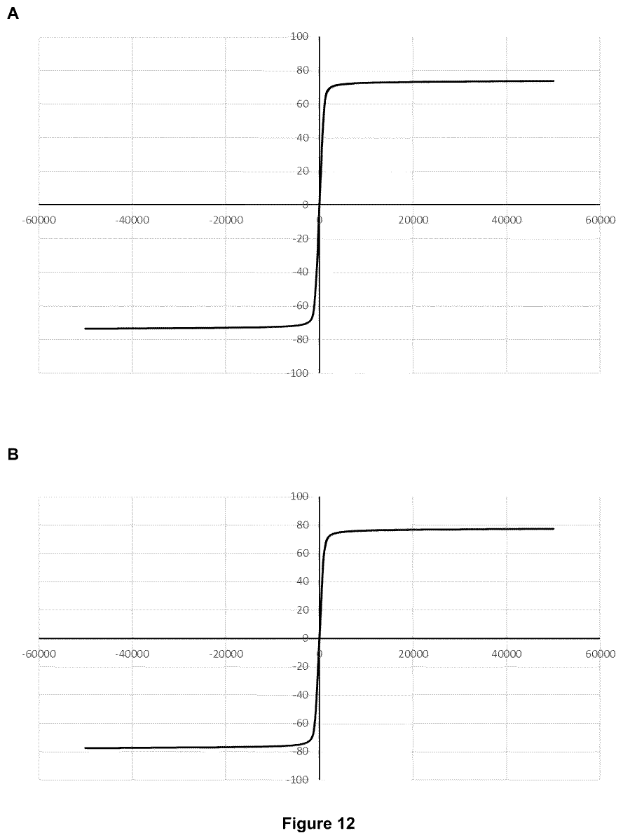

Magnetic hysteresis curves were obtained for the magnetic clusters produced in Example 5 using a magnetic saturation of 72.64 Am²/kg, and for magnetic clusters produced in Example 10 using a magnetic saturation of 72.27 Am²/kg. The hysteresis curves are provided in FIG. 12, with (A) showing results for particles produced using bisphenyl-4-carboxylic acid as the stabilizer (Example 5) and (B) showing results for particles produced using 4-tertbutylbenzoic acid as the stabilizer (Example 10).

The magnetic susceptibility focuses on the part of the hysteresis curve where the magnetizing force decreases from the magnetic saturation point. A value for the magnetic susceptibility can be derived by taking the gradient of the slope around the point the slope intercepts the y axis. Based on the measurements in FIG. 12A, the magnetic clusters produced using bisphenyl-4-carboxylic acid (Example 5) had a magnetic susceptibility of 80.55×10⁻⁵ m³/kg. Based on the measurements from FIG. 12B, the magnetic clusters produced using 4-tertbutylbenzoic acid (Example 10) had a magnetic susceptibility of 120.39×10⁻⁵ m³/kg.

1. Magnetic clusters, wherein the magnetic clusters comprise nanocrystals of iron oxide and an aromatic carboxylic acid.

2. The magnetic clusters of claim 1, wherein the aromatic carboxylic acid is a compound of formula (I): 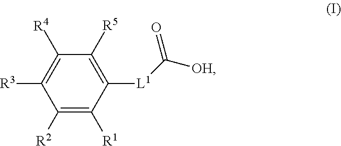
wherein:
L¹ is selected from a bond, —C₁₋₆alkyl-, or —C₂₋₆alkenyl-;
R¹ is selected from —H, —COOH, —(C₁₋₅alkyl)COOH, —(C₂₋₅alkenyl)COOH, or —C₁₋₄alkoxy;
R² is selected from —H, -halo, —C₁₋₄alkyl, —C₁₋₄alkoxy, or —NO₂;
R³ is selected from —H, -halo, —C₁₋₁₂alkyl, —C₃₋₈cycloalkly, —C1-12alkoxy, -halo(C₁₋₁₂)alkyl, —NR⁶R⁷, —NO₂, —C(O)OH, —C(O)OR⁶, or substituted or unsubstituted phenyl;
R⁴ is selected from —H, -halo, —C₁₋₄alkyl, —C₁₋₄alkoxy, or —NO₂;
R⁵ is selected from —H, or —C₁₋₄alkoxy;
R⁶ is selected from —H, or —C₁₋₄alkyl; and
R⁷ is selected from —H, —C₁₋₄alkyl, —C(O)H, —C(O)C₁₋₄alkyl; or
R₁ and R₂ together with the carbon atoms to which they are attached form a substituted or unsubstituted phenyl, and Lt, R³, R⁴, R⁵, R⁶ and R⁷ are as defined previously; or
R₂ and R₃ together with the carbon atoms to which they are attached form a substituted or unsubstituted phenyl, and Lt, Rt, R⁴, R⁵, R⁶ and R⁷ are as defined previously.

3. The magnetic clusters of claim 2, wherein L¹ is selected from a bond, —C₁₋₄alkyl-, or —C₂₋₄alkenyl-.

4. The magnetic clusters of claim 2 or claim 3, wherein L¹ is a bond.

5. The magnetic clusters of claim 2, wherein L¹ is selected from —C₁₋₄alkyl, or —C₂₋₄alkenyl; optionally wherein L¹ is a —C₂₋₄alkenyl-.

6. The magnetic clusters of any of claims 2 to 5, wherein:
R₁ and R₂ together with the carbon atoms to which they are attached form a substituted or unsubstituted phenyl; or
R₂ and R₃ together with the carbon atoms to which they are attached form a substituted or unsubstituted phenyl.

7. The magnetic clusters of any of claims 2 to 5, wherein R¹ is selected from —H, —COOH, —(C₁₋₅alkyl)COOH, or —(C₂₋₅alkenyl)COOH.

8. The magnetic clusters of any of claims 2 to 5 or 7, wherein R¹ is selected from —H, or —COOH.

9. The magnetic clusters of any of claims 2 to 5, 7 or 8, wherein R² is selected from —H, —C₁₋₄alkyl, or —C₁₋₄alkoxy.

10. The magnetic clusters of any of claims 2 to 5 or 7 to 9, wherein R² is selected from —H, or —C₁₋₄alkoxy.

11. The magnetic clusters of any of claims 2 to 5 or 7 to 10, wherein R² is selected from —H, or —OCH₃.

12. The magnetic clusters of any of claims 2 to 5 or 7 to 11, wherein R³ is selected from —H, -halo, —C₁₋₆alkyl, —C₁₋₆alkoxy, -halo(C₁₋₆)alkyl, —NR⁶R⁷, or substituted or unsubstituted phenyl;

13. The magnetic clusters of any of claims 2 to 5 or 7 to 12, wherein R³ is selected from —H, —C₁₋₄alkyl, —C₁₋₄alkoxy, —NR⁶R⁷, or substituted or unsubstituted phenyl;
optionally wherein R³ is selected from —C₁₋₄alkoxy, —NR⁶R⁷, or substituted or unsubstituted phenyl.

14. The magnetic clusters of any of claims 2 to 13, wherein R⁶ is —H or —CH₃, and/or wherein R⁷ is —CH₃ or —C(O)CH₃.

15. The magnetic clusters of any of claims 2 to 5 or 7 to 14, wherein R³ is selected from —H, —C₁₋₄alkyl, —OCH₃, —N(CH₃)₂, —NHC(O)CH₃, or phenyl;
optionally wherein R³ is selected from —OCH₃, —N(CH₃)₂, —NHC(O)CH₃, or phenyl.

16. The magnetic clusters of any of claims 2 to 15, wherein R⁴ is selected from —H, —C₁₋₄alkyl, or —C₁₋₄alkoxy.

17. The magnetic clusters of any of claims 2 to 16, wherein R⁴ is selected from —H, or —C₁₋₄alkoxy.

18. The magnetic clusters of any of claims 2 to 17, wherein R⁴ is selected from —H, or —OCH₃.

19. The magnetic clusters of any of claims 2 to 18, wherein not more than one of R², R³ and R⁴ is a -halo.

20. The magnetic clusters of any of claims 2 to 19, wherein R⁵ is —H.

21. The magnetic clusters of any of claims 2 to 20, wherein R¹ and R⁵ are both —H.

22. The magnetic clusters of any of claims 2 to 21, wherein R³ is —OH, R² is —C₁₋₄alkoxy, and R³ is —C₁₋₄alkoxy.

23. The magnetic clusters of any preceding claim, wherein the aromatic carboxylic acid is selected from benzoic acid, toluic acid (e.g. p-toluic acid), 4-ethylbenzoic acid, 3,5-dimethylbenzoic acid, 4-t-butylbenzoic acid, p-anisic acid, 3,4,5-trimethoxybenzoic acid (eudesmic acid), trans-cinnamic acid, phthalic acid, homophthalic acid, 4-(trifluormethyl)benzoic acid, 4-iodobenzoic acid, 4-chlorobenzoic acid, 3-phenylpropionic acid, 4-(dimethylamino)benzoic acid, 4-acetamidobenzoic acid, bisphenyl-4-carboxylic acid, Methyl hydrogen terephthalate, 3-dimethylaminobenzoic acid, 4-methyl-3,5-dinitrobenzoic acid, 3-bromo benzoic acid, 4-aminobenzoic acid, 4-dimethylaminobenzoic acid, 4-(decycloxy)benzoic acid, 3-bromobenzoic acid, 1-naptholic acid, 4-dimethylaminobenzoic acid, 4-nitrobenzoic acid, terephthalic acid (and/or disodium terephthalate), 4-bromobenzoic acid, 3,5-dimethyl-p-anisic acid, 4-cycloxybenzoic acid, 4-n-pentylbenzoic acid, 4-n-heptylbanzoic acid, 4-n-hexylbenzoic acid, 4-n-butylbenzoic acid, and 2-naptholic acid.

24. The magnetic clusters of any preceding claim, wherein the aromatic carboxylic acid is selected from 4-dimethylaminobenzoic acid, 4-nitrobenzoic acid, terephthalic acid (and/or disodium terephthalate), 4-bromobenzoic acid, 3,5-dimethyl-p-anisic acid, 4-cycloxybenzoic acid, 4-n-pentylbenzoic acid, 4-n-heptylbanzoic acid, 4-n-hexylbenzoic acid, 4-n-butylbenzoic acid, 2-naptholic acid.

25. The magnetic clusters of any proceeding claim, wherein the magnetic clusters are monodisperse.

26. The magnetic clusters of any preceding claim, wherein the magnetic clusters have a mode diameter in the range of from about 100 nm to about 2,500 nm;
optionally wherein the magnetic clusters have a mode diameter in the range of from about 200 nm to about 1,000 nm.

27. Monodisperse magnetic clusters comprising nanocrystals of iron oxide, wherein the magnetic clusters have a mode diameter in the range of from about 100 nm to about 2,500 nm and a coefficient of variation (CV) of not more than 5% when measured by disc centrifuge.

28. The monodisperse magnetic clusters of claim 27, wherein the magnetic clusters further comprise an aromatic carboxylic acid, optionally wherein the aromatic carboxylic acid is as defined in any of claims 2 to 24.

29. The magnetic clusters of any of claims 1 to 25, or monodisperse magnetic clusters of claim 27 or claim 28, wherein the nanocrystals of iron oxide comprise a mode average diameter of from about 5 nm to about 20 nm, optionally wherein the nanocrystals have a mode average diameter of from about 10 nm to about 15 nm, further optionally wherein the nanocrystals have a mode average diameter of about 12 nm.

30. The magnetic clusters of any of claims 1 to 25 or 29, or monodisperse magnetic clusters of any of claims 27 to 29, further comprising a coating.

31. The magnetic clusters or monodisperse magnetic clusters of claim 30, wherein the coating comprises at least one layer of silica.

32. The magnetic clusters or monodisperse magnetic clusters of claim 30 or claim 31, wherein the coating comprises at least one layer of organic coating, optionally wherein the at least one layer of organic coating overcoats at least one layer of silica.

33. The magnetic clusters or monodisperse magnetic clusters of any of claims 30 to 32, wherein the coating comprises functional groups;
optionally wherein the functional groups are selected from hydroxyl groups, carboxylic acid groups, aldehyde groups, amine groups, thiol groups, epoxy groups, amide groups, chloromethyl groups, and tosyl-activated groups;
further optionally wherein the functional groups are selected from a hydroxyl, a carboxylic acid (—COOH), a primary amine, a secondary amine, or epoxy; further optionally wherein the functional groups are selected from a carboxylic acid (—COOH), a primary amine, a secondary amine, or an epoxy.

34. A method of making magnetic clusters, comprising:
forming a solution comprising iron (III) ions, a protic solvent, an aromatic carboxylic acid, and a precipitator; and
allowing the solution to react to form a suspension comprising magnetic clusters.

35. The method of claim 34, wherein the iron (III) ions are provided as a soluble iron (III) salt, optionally wherein the soluble iron (III) salt is an iron (III) halide, such as an iron (III) chloride salt.

36. The method of claim 34 or claim 35, wherein the protic solvent is alcohol, optionally a diol or polyol, further optionally wherein the alcohol is ethylene glycol.

37. The method of any of claims 34 to 36, wherein the aromatic carboxylic acid is as defined in any of claims 2 to 24.

38. The method of any of claims 34 to 37, wherein the precipitator comprises at least one of an acetate, benzoate, or urea, optionally in admixture with an alkali metal halide salt.

39. The method of any of claims 34 to 38, wherein the precipitator comprises sodium acetate, sodium benzoate or urea, optionally in admixture with sodium chloride.

40. The method of any of claims 34 to 38, wherein the precipitator is an acetate, optionally sodium acetate.

41. The method of any of claims 34 to 40. wherein allowing the solution to react comprises one or more of the following conditions:
(a) an elevated temperature, optionally of from 150 °C to 250 °C; and/or
(b) a pressure of from about 1 bar to about 15 bar; and/or
(c) a reaction time of from about 6 h to about 36 h.

42. The method of any of claims 34 to 41, further comprising separating the magnetic clusters from the suspension.

43. The method of claim 42, further comprising washing the magnetic clusters.

44. The method of claim 42 or claim 43, further comprising adding a silica coating to the separated magnetic clusters, comprising:
forming a suspension comprising the separated magnetic clusters and silicates or orthosilicates; and
reacting the silicates or orthosilicates to form a silica coating.

45. The method of claim 44, wherein reacting the silicates to form a silica coating comprises lowering the pH of the suspension to less than 11.

46. The method of claim 44, wherein reacting the orthosilicates to form a silica coating comprises raising the temperature of the suspension to in excess of 40 °C, optionally to in excess of 60 °C or 90 °C.

47. The method of any of any of claims 44 to 46, further comprising overcoating the silica coating with an organic coating, optionally wherein the organic coating comprises a spacer and/or a polymer;
further optionally wherein the overcoating is performed in the presence of Cyrene™.

48. The method of any of claims 34 to 47, wherein the coating comprises functional groups;
optionally wherein the functional groups are selected from hydroxyl groups, carboxylic acid groups, aldehyde groups, amine groups, thiol groups, epoxy groups, amide groups, chloromethyl groups, and tosyl-activated groups;
further optionally wherein the functional groups are selected from a hydroxyl, a carboxylic acid (—COOH), a primary amine, a secondary amine, or epoxy; further optionally wherein the functional groups are selected from a carboxylic acid (—COOH), a primary amine, a secondary amine, or an epoxy.

49. The method of any of claims 34 to 48, wherein the magnetic clusters comprise nanocrystals of iron oxide comprising an average diameter of from about 5 nm to about 40 nm, optionally wherein the nanocrystals have a diameter of from about 5 nm to about 20 nm, further optionally wherein the nanocrystals have a diameter of from about 10 nm to about 15 nm, still further optionally wherein the nanocrystals have a diameter of about 12 nm.

50. The method of any of claims 34 to 49, wherein the magnetic clusters are monodisperse.

51. The method of any of claims 34 to 50, wherein the magnetic clusters have a diameter in the range of from about 100 nm to about 2,500 nm;
optionally wherein the magnetic clusters have a diameter in the range of from about 200 nm to about 1,000 nm.

52. Magnetic clusters obtainable by the method of any of claims 34 to 51.

53. Use of magnetic clusters of any of claims 1 to 33, or of claim 52 in an assay.

54. Use of magnetic clusters of any of claims 1 to 33, or of claim 52 for isolating, purifying, fractionating, depleting, concentrating and/or analysing cells, viruses, vesicles, organelles, exosomes, small molecules, analytes cations, anions, ions, toxins, pathogens, nucleic acids, proteins or peptides.

55. The use of claim 53 or claim 54, wherein the magnetic clusters comprise a (surface) capture ligand, optionally wherein the capture ligand is selected from the group consisting of antibodies, antibody fragments, peptides, carbohydrates, haptens, aptamers, and oligonucleotides.

56. The use of claim 53 or claim 54, wherein the magnetic clusters comprise surface functionalization for capture of a class of molecules, optionally wherein the surface functionalization comprises ionic groups or hydrophobic groups.

57. The use of any of claim 53 or 55-56, wherein the assay comprises immunoprecipitation, nucleic acid capture, immunoassay, and/or lateral flow assay.

58. A kit, comprising:
magnetic clusters of any of claims 1 to 33, or of claim 52.

59. The kit of claim 58, further comprising one or more buffers.

60. The kit of claim 59, wherein the one or more buffers comprise a lysis and/or binding buffer, and optionally a washing buffer and/or an elution buffer.

61. The kit of claim 60, wherein the lysis and/or binding buffer may contain one or more of a chaotrope, a precipitating agent such as an alcohol or an ethylene glycol.
